(19) United States
(12) Patent Application Publication
(10) Pub. No.: US 2025/0075234 A1
(43) Pub. Date: Mar. 6, 2025

(54) COMPOSITIONS AND METHODS FOR BASE EDITING KINASE GENES

(71) Applicant: The Trustees of Columbia University in the City of New York, New York, NY (US)

(72) Inventor: Neil Vasan, Long Island City, NY (US)

(21) Appl. No.: 18/824,298

(22) Filed: Sep. 4, 2024

Related U.S. Application Data
(60) Provisional application No. 63/580,802, filed on Sep. 6, 2023.

(51) Int. Cl. C12N 15/90; C12N 9/22; C12N 9/78; C12N 15/11; C12Q 1/6876

(52) U.S. Cl. CPC C12N 15/907; C12N 9/22; C12N 9/78; C12N 15/11; C12Q 1/6876; C12Y 305/04001; C12Y 305/04002; C07K 2319/00; C12N 2310/20; C12Q 2600/136; C12Q 2600/158

(57) ABSTRACT

A method of modifying a nucleotide in a kinase gene on a double-stranded DNA molecule in a mammalian cell, the method comprising introducing to the cell a composition comprising a guide RNA molecule comprising a spacer sequence portion and a fusion protein comprising a Clustered Regularly Interspaced Short Palindromic Repeats (CRISPR) nuclease and a base editing enzyme.

### CROSS-REFERENCE TO RELATED APPLICATIONS

This application claims the benefit of U.S. Provisional Application No. 63/580,802 filed Sep. 6, 2023, the content of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “240904_92259-A_Sequence_Listing_AWG.xml”, which is 8,845,130 bytes in size, and which was created on Sep. 3, 2024 in the IBM-PC machine format, having an operating system compatibility with MS-Windows, which is contained in the XML file filed Sep. 4, 2024 as part of this application.

### REFERENCE TO LARGE TABLES

This application incorporates-by-reference a large table which is present in the file named “240904_92259-A_Catalytically_Inactive_Kinase_Guides_Table_AWG.txt”, which is 1,945 kilobytes in size, and which was created on Aug. 22, 2024 in the IBM-PC machine format, having an operating system compatibility with MS-Windows, which is contained in the text file filed Sep. 4, 2024 as part of this application.

LENGTHY TABLES
The patent application contains a lengthy table section. A copy of the table is available in electronic form from the USPTO web site (https://seqdata.uspto.gov/?pageRequest=docDetail&DocID=US20250075234A1). An electronic copy of the table will also be available from the USPTO upon request and payment of the fee set forth in 37 CFR 1.19(b)(3).

### TECHNICAL FIELDS

This application relates to compositions and methods for base editing any kinase in the human genome. The compositions and methods may be used to generate screens and cell lines for use in identifying treatments for a variety of conditions, including cancer. Specifically, kinase proteins are druggable enzymes that regulate nearly all aspects of cell signaling in cancer cells, but the ideal kinase targets or kinase drug combinations to kill various types of cancers have not been established. A platform to base edit all 556 human kinases and remove their enzyme activity at physiologic levels is described herein, and may be utilized to decipher the functional effects of how kinase inhibition alters cell growth, drug response, and gene expression which results in accelerated kinase target validation and enable drug discovery. In addition to their uses in screening and cell line generation, the compositions and methods themselves may also be used to treat a condition of a subject, including cancer.

### BACKGROUND OF THE INVENTION

The human genome contains 556 kinase genes, commonly called the “kinome.” Kinases are enzymes that regulate nearly all aspects of cell signaling and their dysregulation leads to excess cell proliferation, a hallmark of cancer. Kinases are one of the most important targets in precision oncology, but the ideal kinase targets or kinase drug combinations for most cancers have not been established. Determining the cellular consequences of turning off kinase enzymes has immense potential to transform our understanding of kinases and kinase inhibitors.

### SUMMARY OF THE INVENTION

According to an embodiment of the present invention, there is provided a method of method of modifying a nucleotide in a kinase gene on a double-stranded DNA molecule in a mammalian cell so as to modify kinase catalytic activity, the method comprising introducing to the cell a composition comprising a guide RNA molecule comprising a spacer sequence portion comprising any one of SEQ ID NOs: 1-10012 and a fusion protein comprising a Clustered Regularly Interspaced Short Palindromic Repeats (CRISPR) nuclease and a base editing enzyme. Preferably, the kinase gene is modified such that the cell expresses a catalytically inactivated form of the kinase having a mutation in its active site.

According to another embodiment of the present invention, there is provided a composition comprising a guide RNA molecule comprising a spacer sequence portion, wherein the spacer sequence portion comprises any one of SEQ ID NOs: 1-10012.

According to another embodiment of the present invention, there is provided a composition comprising a CRISPR guide RNA library, the library comprising a plurality of guide RNA molecules, or a plurality of DNA molecules encoding the guide RNA molecules, wherein each guide RNA molecule comprises a spacer sequence portion, and wherein the library comprises each sequence of SEQ ID NOs: 1-10012 among the spacer sequence portions of the plurality of guide RNA molecules.

### BRIEF DESCRIPTION OF THE DRAWINGS

FIG. 1: Base-editing enzymes to enable drug discovery. Base-editing methods described herein may be used to surgically mutate catalytic amino acids and turn off enzyme catalysis (kinome shown here) in cancer cells, to establish causal relationships between enzyme function and cancer phenotypes that can be therapeutically exploited.

FIGS. 2A-2B: Base editor validation. (FIG. 2A) ABE8e-SpRY-Cas9 base editor construct. Specifically, a lentiviral plasmid construct comprised of 1) TadA** or ABE8e (V106W) (See M. F. Richter et al., Nat Biotechnol. 38, 883-891 (2020)), 2) SpRY-Cas9, a “near PAM-less” Cas9 derivative (Sec R. T. Walton, K. A. Christie, M. N. Whittaker, B. P. Kleinstiver, Science. 368, 290-296 (2020)), 3) eGFP flanked by P2A sites, and 4) a blasticidin resistance cassette. (FIG. 2B) ABE8e-SpRY-Cas9 is a functional base editor, with 93% loss of CD47 in T47D cells, as detected by flow cytometry (light blue and green peaks), using a gRNA mutating CD47 splice sites.

FIGS. 3A-3C: Kinase protein engineering showing (FIG. 3A) kinase catalytic motifs, (FIG. 3B) consequences of adenine base-editing on catalytic amino acids, and (FIG. 3C) identified catalytic sites for 556 kinases in the human genome.

FIGS. 4A-4B: Base editing kinase catalytic function in T47D cells. (FIG. 4A) Experimental schematic. ABE, adenine base editor. (FIG. 4B) Log fold change of control and essential guides.

FIG. 5: Base editing enzyme catalytic function in T47D cells. Volcano plot of pilot base editing experiment in T47D breast cancer cells of kinases. Statistically significant gRNAs showing lethality are in table at bottom. Essential catalytic dead guides, red. Other enzyme guides, black. Negative control guides, gray. Guides labeled by catalytic site and numbered. LFC, log fold change.

FIGS. 6A-6C: Confirmation of base editing in cells for selected PLK4 catalytically dead guide. (FIG. 6A) Pie chart showing proportion of modified reads. (FIG. 6B) Edited sequences and proportions, showing on target catalytic lysine edits and additional edits within the editing window. (FIG. 6C) Nucleotide substitution frequency.

FIG. 7: Base-editing the cancer kinome to enable drug discovery. The approaches described herein bring mutagenesis to genomic scales by utilizing base-editing methods to surgically mutate catalytic amino acids and turn off kinome catalysis in cancer cells, to establish causal relationships between kinase function and cancer phenotypes that can be therapeutically exploited.

FIGS. 8A-8B: Adenine base-editing schematic showing (FIG. 8A) adenine base-editing mechanism and (FIG. 8B) cloned plasmid featuring an adenine base editor that can bind to any sequence for site-specific editing.

FIGS. 9A-9C: Kinase protein engineering showing (FIG. 9A) kinase catalytic motifs, (FIG. 9B) consequences of adenine base-editing on catalytic amino acids, and (FIG. 9C) identified catalytic sites for 556 kinases in the human genome.

FIG. 10: Kinome base-editing gRNA library targeting catalytic residues (kinase-dead) and splice sites (knock-out), and controls for base editing all 556 human kinases.

FIG. 11: Phosphoproteomic validation of on-target effects of kinase-dead mutants based on a global atlas of substrate specificities for the human kinome (See Needham, EJ et al. Sci Signal 12 (2019)).

FIG. 12: Schematic for kinase catalytic base-editing in pooled cellular assays. ABE=adenine base editor.

FIG. 13: Drugging strategies based on kinase-dead and kinase knock-out effects on cancer cell lethality.

FIG. 14: Kinase-druggable gene interdependency network using single-cell RNA sequencing to determine the druggable genes whose expression is altered by kinase catalytic inhibition.

FIG. 15: Overview of base editing screen design. Target cells are first transduced with ABE construct (see FIG. 2A) and selected with blasticidin and then transduced with guide RNA library at low MOI followed by puromycin selection. After that D0 cell sample collected and cells maintained for additional 2 weeks before collecting second D14 sample. Genomic DNA was isolated from D0 and D14 samples followed by next generation sequencing to get the guide counts. Guide counts were compared between D14 and D0 samples. If gRNA was depleted it indicated that kinase inhibition decreases cell growth and gRNA enrichment indicated that kinase inhibition increases growth.

FIGS. 16A-16B: Screen quality control data. Next generation sequencing of guide counts were trimmed. (FIG. 16A) Log 2 counts for all guides from each sample replicate. (FIG. 16B) Table showing reads mapped and statistics for each replicate. The Gini Index describes the read count distribution. A smaller value indicates more evenness of the count distribution. Next generation sequencing of guide counts trimmed using following packages: Trimming of adapter sequences: cutadapt, see M. Martin, EMBnet.journal. 17, 10-12 (2011); and Guide counts, normalization, and Robust Rank Aggregation (RRA) analysis: MAGeCK-VISPR, see W. Li et al., Genome Biol. 16, 281 (2015) and Wang et al., Nat Protoc. 14, 756-780 (2019).

FIG. 17: Replicate correlations. Correlations between replicates for t0 and t14 samples. Graphs made in R using ggscatter, R=Pearson correlation coefficient.

FIGS. 18A-18B: Screen quality and controls. (FIG. 18A) Log 2 fold changes for guides by guide group (average of three (3) replicates). Ctrl_NT=non-targeting guides, Ctrl_ESS=splice site guides targeting common essential genes (non-kinase), Ctrl_NONESS=splice site guides targeting non-essential genes, Ctrl_EW=guides without adenines in ABE editing window, Kinase_Cat=guides targeting catalytic residues in kinases, Kinase_splice=guides targeting splice sites in kinases. (FIG. 18B) Statistical significances between all of the guide groups calculated using Brown-Forsythe and Welch ANOVA test with Games-Howell multiple test correction.

FIG. 19: Screen data. Volcano plot showing kinase hits. Each dot corresponds to one catalytic site residue (average of all guides targeting that site from three (3) replicates). Cut-off values indicated with grey dotted lines: FDR<10%, LFC<−0.6 or >0.6.

### DETAILED DESCRIPTION

In order to facilitate an understanding of the subject matter disclosed herein, each of the following terms, as used herein, shall have the meaning set forth below, except as expressly provided otherwise herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, all numerical ranges provided are intended to expressly include at least the endpoints and all numbers that fall between the endpoints of ranges.

As used herein, the term “targeting sequence” or “targeting molecule” in the context of guide RNA molecules refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of a guide RNA molecule that can form a complex with a CRISPR nuclease, either alone or in combination with another RNA molecule (e.g. a tracrRNA molecule), with the targeting sequence serving as the targeting portion of the CRISPR complex. When the guide RNA molecule having the targeting sequence is present contemporaneously with the CRISPR molecule, the guide RNA molecule, alone or in combination with an additional one or more RNA molecules (e.g. a tracrRNA molecule), is capable of targeting the CRISPR nuclease to the specific target sequence. As non-limiting example, a spacer sequence portion of a CRISPR RNA (crRNA) molecule or single-guide RNA (sgRNA) molecule may serve as a targeting molecule.

The term “targets” as used herein in the context of guide RNA molecules, refers to preferentially hybridizing a targeting sequence of a targeting molecule to a nucleic acid molecule having a targeted nucleotide sequence. It is understood that the term “targets” encompasses variable hybridization efficiencies, such that there is preferential targeting of the nucleic acid molecule comprising the targeted nucleotide sequence. It is understood that where a guide RNA molecule targets a sequence, a complex of the guide RNA molecule and a CRISPR nuclease targets the sequence for binding and/or nuclease activity.

The “spacer sequence portion” of a guide RNA molecule refers to a nucleotide sequence that is capable of hybridizing to a specific target DNA sequence, e.g., the spacer sequence portion has a nucleotide sequence which is partially or fully complementary to the DNA sequence being targeted along the length of the spacer sequence portion. The entire length of the spacer sequence portion may be complementary, preferably fully or mostly complementary, to the DNA sequence being targeted along the length of the spacer sequence portion. The spacer sequence portion may be part of a guide RNA molecule that can form a complex with a CRISPR nuclease, with the spacer sequence portion serving as the DNA-targeting portion of the complex. When a guide RNA molecule having the spacer sequence portion is present contemporaneously with a CRISPR molecule, alone or in combination with an additional one or more RNA molecules (e.g. a tracrRNA molecule), the guide RNA molecule is capable of targeting the CRISPR nuclease to the specific target DNA sequence. Accordingly, a CRISPR complex can be formed by direct binding of a guide RNA molecule having the spacer sequence portion to a CRISPR nuclease or by binding of the guide RNA molecule having the spacer sequence portion and an additional one or more RNA molecules to the CRISPR nuclease. Each possibility represents a separate embodiment. A spacer sequence portion can be custom designed to target any desired sequence and satisfy a PAM requirement of a CRISPR nuclease. In some embodiments, a spacer sequence portion may comprise any one of SEQ ID NOs: 1-10012. In some embodiments, a spacer sequence portion comprises any one of SEQ ID NOs: 1-10012 and additional nucleotides, e.g. additional nucleotides fully complimentary to a nucleotide or sequence of nucleotides adjacent to the 3′ end of the target sequence, 5′ end of the target sequence, or both. In some embodiments, a spacer sequence portion comprises a portion of any one of SEQ ID NOs: 1-10012 with 1, 2, 3, 4, or 5 nucleotides removed from one or both ends of the sequence. In some embodiments, the spacer sequence portion comprises a sequence that is the same as, or differs by no more than 1, 2, 3, 4, or 5 nucleotides from, a spacer sequence portion of any one of SEQ ID NOs: 1-10012.

Spacer sequence portions which target regions encoding catalytically active sites of kinases are provided in the large table in the file named “240904_92259-A_Catalytically_Inactive_Kinase_Guides_Table_AWG.txt”. The targeted kinase gene is listed in column 1. A name for each spacer sequence portion, which includes the targeted kinase catalytic site motif, is listed in column 2. The chromosome of the targeted kinase is provided in column 3. The start and end positions of a codon encoding an amino acid present in the targeted kinase motif, which can be modified using the spacer sequence portion, is provided in columns 4 and 5, respectively. For example, the space sequence portion named “AAK1_DFG_1” may be used to modify a nucleotide in the AAK1 gene which encodes the aspartic acid residue (D) in the DFG catalytic site motif of the AAK1 kinase. The strand of the genome containing a sequence corresponding to the spacer sequence is provided in column 6. The EnsemblP_ID of the kinase is provided in column 7. Spacer sequence portions which may be used according to the methods described herein to modify a region encoding a kinase catalytic site are provided in column 8, along with their respective SEQ ID NOs.

Accordingly, a molecule comprising a “spacer sequence portion” is a type of targeting molecule. Throughout this application, the terms “guide molecule,” “RNA guide molecule,” “guide RNA molecule,” and “gRNA molecule” are synonymous with a molecule comprising a spacer sequence portion. Guide RNA molecules and their uses as sgRNA molecules or in crRNA:tracrRNA complexes are well-understood in the art. See, for example, Jinek et al. “A programmable dual-RNA-guided DNA endonuclease in adaptive bacterial immunity” Science, 17; 337(6096): 816-21 (2012).

A skilled artisan will appreciate that a guide RNA molecule can be engineered to bind to a desired target sequence in a genome by commonly known methods in the art. Specifically, an engineered guide RNA molecule may be designed such as to associate with a target genomic DNA sequence of interest in proximity to a protospacer adjacent motif (PAM), e.g., a PAM compatible with the CRISPR nuclease utilized with the guide RNA molecule. As non-limiting examples, a guide RNA molecule may be engineered to target a sequence next to a PAM of NGG or NAG, wherein “N” is any nucleobase, for use with Streptococcus pyogenes Cas9 WT (SpCas9); or next to a PAM of NR, NRN, or NYN, wherein N is any nucleobase, R is A or G and Y is C or T, for use with an SpRY variant of SpCas9. Additional CRISPR nuclease orthologues from various species, and variants thereof, are known in the art, as well as their PAM requirements. See for example, Ren et al., “PAM-less plant genome editing using a CRISPR-SpRY toolbox” Nature Plants, 7, 25-33 (2021); Ye et al. “Can SpRY recognize any PAM in human cells?” J. Zheijiang Univ. Sci. B., 23 (5): 382-391 (2022); and Liang et al. “SpG and SpRY variants expand the CRISPR toolbox for genome editing in zebrafish” Nature Communications, 13, 3421 (2022).

Accordingly, guide RNA molecules described herein are designed to form complexes in conjunction with one or more different CRISPR nucleases and to target polynucleotide sequences of interest utilizing one or more different PAM sequences respective to the CRISPR nuclease utilized.

As a non-limiting example, a CRISPR nuclease derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence may be utilized in embodiments of the invention.

The following embodiments and examples (including details thereof) are set forth to aid in an understanding of the subject matter of this disclosure but are not intended to, and should not be construed to, limit in any way the invention that is claimed.

According to embodiments of the present invention, there is provided a method of modifying a nucleotide in a kinase gene on a double-stranded DNA molecule in a mammalian cell, the method comprising introducing to the cell a composition comprising a guide RNA molecule comprising a spacer sequence portion and a fusion protein comprising a Clustered Regularly Interspaced Short Palindromic Repeats (CRISPR) nuclease and a base editing enzyme. Preferably, a nucleotide within a region encoding an active site of the kinase is modified such that the kinase expressed from the modified gene is catalytically inactive. Accordingly, the method results in a modified kinase gene that encodes a catalytically inactive kinase. Thus, a full-length, catalytically inactive kinase may be expressed from the modified kinase gene.

In some embodiments, the base editing enzyme is an adenine deaminase.

In some embodiments, the base editing enzyme is ABE8e.

In some embodiments, the base editing enzyme is a cytosine deaminase, and the fusion protein preferably further comprises an inhibitor of uracil DNA glycosylase (UGI).

In some embodiments, the CRISPR nuclease is a nickase and effects a single-strand break in a strand of the double-stranded DNA molecule, preferably in a strand that the spacer sequence portion of the guide RNA molecule is hybridized to.

In some embodiments, the CRISPR nuclease is a nickase comprising an inactive HNH domain.

In some embodiments, the CRISPR nuclease is catalytically inactive.

In some embodiments, the CRISPR nuclease is a SpCas9, SaCas9, SpRY-Cas9, or an orthologue and/or variant thereof.

In some embodiments, the CRISPR nuclease is SpRY-Cas9.

In some embodiments, the base editing enzyme is N-terminal of the CRISPR nuclease in the fusion protein.

In some embodiments, the fusion protein comprises a linker portion between the CRISPR nuclease and the base editing enzyme, preferably wherein the linker portion is a rigid or flexible linker.

In some embodiments, the kinase gene is selected from the group consisting of AAK1, AATK, ABL1, ABL2, ACAD10, ACAD11, ACVR1, ACVR1B, ACVR1C, ACVR2A, ACVR2B, ACVRL1, ADCK1, ADCK2, ADCK5, AKT1, AKT2, AKT3, ALK, ALPK1, ALPK2, ALPK3, AMHR2, ANKK1, ARAF, ATM, ATR, AURKA, AURKB, AURKC, AXL, BLK, BMP2K, BMPR1A, BMPR1B, BMPR2, BMX, BRAF, BRSK1, BRSK2, BTK, BUB1, BUB1B, CASMK1, CASMK1D, CAMK1G, CAMK2A, CAMK2B, CAMK2D, CAMK2G, CAMK4, CAMKK1, CAMKK2, CAMKV, CASK, CDC42BPA, CDC42BPB, CDC42BPG, CDC7, CDK1, CDK10, CDK11A, CDK11B, CDK12, CDK13, CDK14, CDK15, CDK16, CDK17, CDK18, CDK19, CDK2, CDK20, CDK3, CDK4, CDK5, CDK6, CDK7, CDK8, CDK9, CDK1, CDKL2, CDKL3, CDKL4, CDKL5, CHEK1, CHEK2, CHKA, CHKB, CHUK, CILK1, CIT, CLK1, CLK2, CLK3, CLK4, COQ8A, COQ8B, CSF1R, CSK, CSNK1A1, CSNK1A1L, CSNK1D, CSNK1E, CSNK1G1, CSNK1G2, CSNK1G3, CSNK2A1, CSNK2A2, CSNK2A3, DAPK1, DAPK2, DAPK3, DCLK1, DCLK2, DCLK3, DDR1, DDR2, DMPK, DSTYK, DYRK1A, DYRK1B, DYRK2, DYRK3, DYRK4, EEF2K, EGFR, EIF2AK1, EIF2AK2, EIF2AK3, EIF2AK4, EPHA1, EPHA2, EPHA3, EPHA4, EPHA5, EPHA6, EPHA7, EPHA8, EPHB1, EPHB2, EPHB3, EPHB4, ERBB2, ERBB3, ERBB4, ERN1, ERN2, ETNK1, ETNK2, FAM20A, FAM20B, FAM20C, FER, FES, FGFR1, FGFR2, FGFR3, FGFR4, FGR, FLT1, FLT3, FLT4, FN3K, FN3KRP, FRK, FYN, GAK, GRK1, GRK2, GRK3, GRK4, GRK5, GRK6, GRK7, GSK3A, GSK3B, GUCY2C, GUCY2D, GUCY2F, HASPIN, HCK, HIPK1, HIPK2, HIPK3, HIPK4, HUNK, HYKK, IGF1R, IKBKB, IKBKE, ILK, INSR, INSRR, IP6K1, IP6K2, IP6K3, IPMK, IPPK, IRAK1, IRAK2, IRAK3, IRAK4, ITK, ITPKA, ITPKB, ITPKC, JAK1, JAK2, JAK3, KALRN, KDR, KIT, KSR1, KSR2, LATS1, LATS2, LCK, LIMK1, LIMK2, LMTK2, LMTK3, LRRK1, LRRK2, LTK, LYN, MAK, MAP2K1, MAP2K2, MAP2K3, MAP2K4, MAP2K5, MAP2K6, MAP2K7, MAP3K1, MAP3K10, MAP3K11, MAP3K12, MAP3K13, MAP3K14, MAP3K15, MAP3K19, MAP3K2, MAP3K20, MAP3K21, MAP3K3, MAP3K4, MAP3K5, MAP3K6, MAP3K7, MAP3K8, MAP3K9, MAP4K1, MAP4K2, MAP4K3, MAP4K4, MAP4K5, MAPK1, MAPK10, MAPK11, MAPK12, MAPK13, MAPK14, MAPK15, MAPK3, MAPK4, MAPK6, MAPK7, MAPK8, MAPK9, MAPKAPK2, MAPKAPK3, MAPKAPK5, MARK1, MARK2, MARK3, MARK4, MAST1, MAST2, MAST3, MAST4, MASTL, MATK, MELK, MERTK, MET, MINK1, MKNK1, MKNK2, MLKL, MOK, MOS, MST1R, MTOR, MUSK, MYLK, MYLK2, MYLK3, MYLK4, MYO3A, MYO3B, NEK1, NEK10, NEK11, NEK2, NEK3, NEK4, NEK5, NEK6, NEK7, NEK8, NEK9, NIM1K, NLK, NPR1, NPR2, NRK, NTRK1, NTRK2, NTRK3, NUAK1, NUAK2, OBSCN, OXSR1, PAK1, PAK2, PAK3, PAK4, PAK5, PAK6, PASK, PBK, PDGFRA, PDGFRB, PDIK1L, PDPK1, PEAK1, PEAK3, PHKG1, PHKG2, PI4K2A, PI4K2B, PI4KA, PI4KB, PIK3C2A, PIK3C2B, PIK3C2G, PIK3C3, PIK3CA, PIK3CB, PIK3CD, PIK3CG, PIK3R4, PIKFYVE, PIM1, PIM2, PIM3, PINK1, PIP4K2A, PIP4K2B, PIP4K2C, PIP5K1A, PIP5K1B, PIP5K1C, PIP5KL1, PKDCC, PKMYT1, PKN1, PKN2, PKN3, PLK1, PLK2, PLK3, PLK4, PNCK, POMK, PRAG1, PRKAA1, PRKAA2, PRKACA, PRKACB, PRKACG, PRKCA, PRKCB, PRKCD, PRKCE, PRKCG, PRKCH, PRKCI, PRKCQ, PRKCZ, PRKD1, PRKD2, PRKD3, PRKDC, PRKG1, PRKG2, PRKX, PRPF4B, PSKH1, PSKH2, PTK2, PTK2B, PTK6, PTK7, PXK, RAF1, RET, RIOK1, RIOK2, RIOK3, RIPK1, RIPK2, RIPK3, RIPK4, RNASEL, ROCK1, ROCK2, ROR1, ROR2, ROS1, RPS6KA1, RPS6KA2, RPS6KA3, RPS6KA4, RPS6KA5, RPS6KA6, RPS6KB1, RPS6KB2, RPS6KC1, RPS6KL1, RSKR, RYK, SBK1, SBK2, SBK3, SELENOO, SGK1, SGK2, SGK3, SIK1, SIK2, SIK3, SLK, SMG1, SNRK, SPEG, SRC, SRMS, SRPK1, SRPK2, SRPK3, STK10, STK11, STK16, STK17A, STK17B, STK24, STK25, STK26, STK3, STK31, STK32A, STK32B, STK32C, STK33, STK35, STK36, STK38, STK38L, STK39, STK4, STK40, STKLD1, STYK1, SYK, TAOK1, TAOK2, TAOK3, TBCK, TBK1, TEC, TEK, TESK1, TESK2, TEX14, TGFBR1, TGFBR2, TIE1, TLK1, TLK2, TNIK, TNK1, TNK2, TNNI3K, TP53RK, TRIB1, TRIB2, TRIB3, TRIO, TRPM6, TRPM7, TSSK1B, TSSK2, TSSK3, TSSK4, TSSK6, TTBK1, TTBK2, TTK, TTN, TXK, TYK2, TYRO3, UHMK1, ULK1, ULK2, ULK3, ULK4, VRK1, VRK2, VRK3, WEE1, WEE2, WNK1, WNK2, WNK3, WNK4, YES1, and ZAP70.

In some embodiments, the kinase gene is selected from the group consisting of ATM, PLK4, and CDK9.

In some embodiments, the base editing enzyme modifies a base of a nucleotide on a coding strand or a non-coding strand of the kinase gene.

In some embodiments, a CRISPR complex comprising the guide RNA molecule and the CRISPR nuclease displace a strand of the DNA molecule, wherein the base editing enzyme modifies a nucleotide base on the displaced strand, and wherein the modified nucleotide is on a coding strand or a non-coding strand of the kinase gene.

In some embodiments, a A:T to G:C conversion or a C:G to T:A conversion occurs in the double-stranded DNA molecule at the site of the modified nucleotide

In some embodiments, cellular DNA mismatch repair is induced by the nucleotide modification and a A:T to G:C conversion or a C:G to T:A conversion occurs in the double-stranded DNA molecule at the site of the modified nucleotide.

In some embodiments, the double-stranded DNA molecule is genomic DNA.

In some embodiments, the mammalian cell is a mouse cell, a non-human primate cell, or a human cell.

In some embodiments, the mammalian cell is a primary cell, a cancer cell, or an immortalized cell.

In some embodiments, the mammalian cell is a HAP1 leukemia cell, MDA-MB-231 (breast) cell, A375 (skin) cell, HT29 (colon) cell, A549 (lung) cell, MCF7 (breast) cell, or a T47D (breast) cell.

In some embodiments, the mammalian cell is a cancer cell obtained from a subject having breast cancer, estrogen receptor positive (ER+) breast cancer, triple-negative breast cancer (TNBC), colon cancer, prostate cancer, bladder cancer, soft-tissue sarcoma, an advanced lung cancer, lung cancer, non-small cell lung cancer, small cell lung cancer, mesothelioma, esophageal cancer, liver cancer, renal cell cancer, melanoma, skin cancer, basal cell skin cancer, squamous cell skin cancer, and squamous cell carcinoma of the head and neck, or leukemia.

In some embodiments, the modified cell expresses a full-length catalytically inactive form of the kinase encoded by the modified kinase gene.

In some embodiments, the modified kinase gene encodes a stable, catalytically inactive form of the kinase comprising an amino acid substitution in VAIK, HRD, or DFG motif.

In some embodiments, the lysine in a VAIK motif is substituted to glutamate, arginine, or glycine.

In some embodiments, the aspartate in a HRD or DFG motif is substituted to glycine.

In some embodiments, the spacer sequence portion comprises any one of SEQ ID NOs: 1-10012.

In some embodiments, the spacer sequence portion comprises SEQ ID NO: 6982.

In some embodiments, the guide RNA molecule is a single guide RNA (sgRNA) molecule.

In some embodiments, the guide RNA molecule is a CRISPR RNA (crRNA) molecule, and the composition further comprises a transactivating RNA (tracrRNA) molecule that hybridizes to a repeat sequence portion of the crRNA molecule to form a crRNA:tracrRNA complex.

In some embodiments, the sgRNA molecule or crRNA:tracrRNA complex are capable of forming a CRISPR complex with the CRISPR nuclease, and wherein the spacer portion of the guide RNA molecule in the CRIPSR complex hybridizes to a strand of the double-stranded DNA molecule.

In some embodiments, the guide RNA molecule and/or tracrRNA molecule are introduced to the cell by introducing a DNA polynucleotide encoding the guide RNA molecule and/or tracrRNA molecule.

In some embodiments, the guide RNA molecule and/or tracrRNA molecule are introduced by directly delivering the guide RNA molecule and/or tracrRNA molecule to the cell.

In some embodiments, the fusion molecule is introduced to the cell by introducing to the cell a polynucleotide encoding the fusion protein.

In some embodiments, the fusion protein is introduced to the cell by directly delivering the fusion protein to the cell.

In some embodiments, the fusion protein is introduced to the cell prior to the introduction of the guide RNA molecule.

In some embodiments, the fusion protein and the guide RNA molecule are introduced at the same time or substantially the same time.

In some embodiments, a CRISPR complex comprising the guide RNA molecule and the CRISPR nuclease is formed and directly introduced into the cell.

In some embodiments, the composition is introduced to a cell ex vivo, in vivo, or in vitro.

In some embodiments, the composition is introduced to a cell isolated from a tumour.

In some embodiments, the composition is introduced to a cell in a subject, preferably a human subject, more preferably to a tumour cell in a human subject.

According to embodiments of the present invention, there is provided a modified mammalian cell obtained by any one of the methods described herein. In some embodiments, a kinase gene has been modified in the modified mammalian cell such that the cell expresses a catalytically inactive form of the kinase, preferably such that an amino acid in the active site of the kinase has been altered relative to a wild-type form of the kinase.

According to embodiments of the present invention, there is provided a composition comprising a guide RNA molecule comprising a spacer sequence portion, wherein the spacer sequence portion comprises any one of SEQ ID NOs: 1-10012.

In some embodiments, the guide RNA molecule is a crRNA molecule or a sgRNA molecule.

In some embodiments, the guide RNA molecule is a crRNA molecule and the composition further comprises a tracrRNA molecule that hybridizes to a repeat sequence portion of the crRNA molecule to form a crRNA:tracrRNA complex.

In some embodiments, the composition further comprises a CRISPR nuclease or a fusion protein comprising a CRISPR nuclease and a base editing enzyme.

In some embodiments, the CRISPR nuclease is a nickase

In some embodiments, the CRISPR nuclease comprises an inactive HNH domain.

In some embodiments, the CRISPR nuclease is catalytically inactive.

In some embodiments, the CRISPR nuclease is a SpCas9, SaCas9, SpRY-Cas9, or an orthologue and/or variant thereof.

In some embodiments, the CRISPR nuclease is SpRY-Cas9.

In some embodiments, the composition further comprises a fusion protein comprising a CRISPR nuclease and a base editing enzyme.

In some embodiments, the base editing enzyme is N-terminal of the CRISPR nuclease in the fusion protein.

In some embodiments, the fusion protein comprises a linker portion between the CRISPR nuclease and the base editing enzyme, preferably wherein the linker portion is a rigid or flexible linker.

In some embodiments, the base editing enzyme is an adenine deaminase.

In some embodiments, the base editing enzyme is ABE8e.

In some embodiments, the base editing enzyme is a cytosine deaminase, and the fusion protein preferably further comprises an inhibitor of uracil DNA glycosylase (UGI).

In some embodiments, the composition is packaged in a viral delivery system, preferably a lentivirus or adeno-associated virus (AAV).

In some embodiments, the spacer sequence portion comprises SEQ ID NO: 6982.

According to embodiments of the present invention, there is provided any one of the compositions described herein for use in treating a condition in a subject, preferably wherein the condition is a cancer.

According to embodiments of the present invention, there is provided a method of treating a condition in a subject comprising delivering any one of the compositions described herein to the subject, preferably wherein the condition is a cancer.

According to embodiments of the present invention, there is provided a polynucleotide molecule encoding any of the guide RNA molecules, crRNA molecules, tracrRNA molecules, sgRNA molecules, CRISPR nucleases, and/or fusion proteins described herein.

According to embodiments of the present invention, there is provided a composition comprising a CRISPR guide RNA library, the library comprising a plurality of guide RNA molecules, or a plurality of DNA molecules encoding the guide RNA molecules, wherein each guide RNA molecule comprises a spacer sequence portion, and wherein the library comprises each sequence of SEQ ID NOs: 1-10012 among the spacer sequence portions of the plurality of guide RNA molecules.

In some embodiments, the library is packaged in a viral delivery system, preferably a lentivirus.

In some embodiments, the library comprises a plurality of DNA molecules encoding the guide RNA molecules.

In some embodiments, the DNA molecules are plasmids or viral vector molecules, preferably lentiviral vector molecules.

In some embodiments, the library comprises a plurality of DNA molecules encoding the guide RNA molecules, and each DNA molecule also encodes a fusion protein comprising a CRISPR nuclease and a base editing enzyme.

In some embodiments, the composition further comprises a fusion protein comprising a CRISPR nuclease and a base editing enzyme or a DNA molecule encoding the fusion protein comprising a CRISPR nuclease and a base editing enzyme.

In some embodiments, the CRISPR nuclease is a nickase.

In some embodiments, the CRISPR nuclease comprises an inactive HNH domain.

In some embodiments, the CRISPR nuclease is catalytically inactive.

In some embodiments, the CRISPR nuclease is a SpCas9, SaCas9, SpRY-Cas9, or an orthologue and/or variant thereof.

In some embodiments, the CRISPR nuclease is SpRY-Cas9.

In some embodiments, the composition further comprises a fusion protein comprising a CRISPR nuclease and a base editing enzyme.

In some embodiments, the base editing enzyme is N-terminal of the CRISPR nuclease in the fusion protein.

In some embodiments, the fusion protein comprises a linker portion between the CRISPR nuclease and the base editing enzyme, preferably wherein the linker portion is a rigid or flexible linker.

In some embodiments, the base editing enzyme is an adenine deaminase.

In some embodiments, the base editing enzyme is ABE8e.

In some embodiments, the base editing enzyme is a cytosine deaminase, and the fusion protein preferably further comprises an inhibitor of uracil DNA glycosylase (UGI).

According to embodiments of the present invention, there is provided a plurality of modified cells, wherein the cells are modified by introducing the any one of the compositions described herein to the cells.

In some embodiments, the fusion protein comprising a CRISPR nuclease and a base editing enzyme is expressed in the cell before or after the CRISPR guide RNA library is introduced.

In some embodiments, the cells are mammalian cells, non-human primate cells, or human cells.

In some embodiments, the cells are primary cells, cancer cells, or an immortalized cells.

In some embodiments, the cells are HAP1 leukemia cells, MDA-MB-231 (breast) cells, A375 (skin) cells, HT29 (colon) cells, A549 (lung) cells, MCF7 (breast) cells, or a T47D (breast) cells.

In some embodiments, the cells are cancer cells obtained from a subject having breast cancer, estrogen receptor positive (ER+) breast cancer, triple-negative breast cancer (TNBC), colon cancer, prostate cancer, bladder cancer, soft-tissue sarcoma, an advanced lung cancer, lung cancer, non-small cell lung cancer, small cell lung cancer, mesothelioma, esophageal cancer, liver cancer, renal cell cancer, melanoma, skin cancer, basal cell skin cancer, squamous cell skin cancer, and squamous cell carcinoma of the head and neck, or leukemia.

According to embodiments of the present invention, there is provided an isogenic cell line generated from a cell isolated from the plurality of modified cells.

According to embodiments of the present invention, there is provided a method of identifying a drug target for use in a combination therapy with a first treatment, the method comprising
i) exposing the plurality of modified cells to the first treatment;
ii) sequencing the spacer sequence portions in the plurality of cells, preferably at a time point 0.5-14 days after the exposure to the first treatment;
iii) determining if each spacer sequence portion was enriched, unchanged, or depleted;
iv) classifying a kinase as a catalytically growth-suppressive kinase if a spacer sequence portion targeting the kinase is enriched, and classifying a kinase as a catalytically essential kinase if a spacer sequence portion targeting the kinase is depleted; and
v) identifying a catalytically essential kinase as a drug target for use in a combination therapy with the first treatment.

According to embodiments of the present invention, there is provided a method of identifying a drug target, the method comprising
i) sequencing the spacer sequence portions in the plurality of modified cells, preferably at a time point 0.5-30 days after the introduction of the CRISPR guide RNA library to the plurality of cells;
ii) determining if each spacer sequence portion was enriched, unchanged, or depleted;
iii) classifying a kinase as a catalytically growth-suppressive kinase if a spacer sequence portion targeting the kinase is enriched, and classifying a kinase as a catalytically essential kinase if a spacer sequence portion targeting the kinase is depleted; and
iv) identifying a catalytically essential kinase as a drug target.

According to embodiments of the present invention, there is provided a method of identifying a drug target, the method comprising
i) sequencing the spacer sequence portions in the plurality of modified cells, preferably at a time point 0.5-30 days after the introduction of the CRISPR guide RNA library to the plurality of cells;
ii) performing single-cell RNA sequencing on a cell of the plurality of modified cells, measuring gene expression levels in the cell; and
iii) identifying a gene whose expression is altered relative to an unmodified cell as a drug target.

In some embodiments, the method further comprises selecting a treatment that affects the drug target,
wherein a kinase degrader molecule is selected as the treatment if
i) a first spacer sequence portion comprising any one of SEQ ID NOs: 1-10012 is enriched or unchanged; and
ii) a second spacer sequence portion which targets and knocks out the same kinase as targeted by the first spacer sequence portion of i) is depleted.

In some embodiments, the method further comprises selecting a treatment that affects the drug target,
wherein an active site kinase inhibitor is selected as the treatment if
i) a first spacer sequence portion comprising any one of SEQ ID NOs: 1-10012 is depleted; and
ii) a second spacer sequence portion which targets and knocks out the same kinase as targeted by the first spacer sequence portion of i) is enriched or unchanged.

In some embodiments, the method further comprises administering a treatment that affects the drug target to a subject.

In some embodiments, the plurality of modified cells are cancer cells and the treatment that affects the drug target is a cancer treatment.

According to embodiments of the present invention, there is provided a composition, method, or modified cell as characterized by one or more elements disclosed herein.

Disclosed herein are compositions and methods to perturb kinase catalysis and measure its effects on cancer cell function. More specifically, the use of cutting-edge base editing tools to precisely mutate amino acids necessary for enzyme catalysis in every human kinase across the kinome is demonstrated. This approach may be utilized to identify novel kinases for therapeutic targeting and act as a surrogate for small-molecule inhibition to enable drug discovery. Prior methods to perturb the kinome in cells use knock-out or knock-down approaches, which are crude tools that alter expression of the entire gene and do not necessarily translate to the effects of a small-molecule inhibitor.

The base editing approach to inactivate kinase catalysis as described herein solves two major issues that prevent existing mutagenesis and knock-in methods to both scale across the kinome and perform mutations at physiologic levels. Base editing can mutate hundreds of genes at physiologic levels in a pooled experiment due to its high editing efficiency. Disclosed herein is a base editing platform to selectively mutate individual catalytic amino acids in 556 kinases in cancer cells for pooled functional studies. As a case study, the platform was used in breast cancer cells, where multiple kinase inhibitors are standard therapies but where new treatments are urgently needed.

The base editing platform includes a base editor that can bind to any genomic sequence for site-specific editing, identification of catalytically necessary amino acids in every human kinase, a guide RNA library to edit amino acids resulting in kinase catalytic loss, and sophisticated proteomic strategies to prove on-target loss of kinase catalysis in cells.

The base editing platform is used to interrogate how kinase activity regulates 1) cancer cell growth, 2) sensitivity to targeted therapies, and 3) druggable gene expression. Notably, this platform will drastically compress the interval between kinase hit and lead discovery from a time scale of decades to years by identifying the optimal mechanisms to inhibit kinases, which is transformative for drug development and the design of combination therapies.

This platform is innovative conceptually, technically, and materially as it provides the first approach for precise, surgical inhibition of a large and diverse family of enzymes in a comprehensive fashion.

As a non-limiting example, the present technology provides compositions and methods for catalytically inactivating kinases. The compositions and methods may be used for generating stable cell lines, screening for new or improved treatments of a condition, or directly used to treat conditions themselves.

Notably, the compositions and methods described herein solve the problem of how to genetically and endogenously model kinase inhibition (and other enzyme inhibition) in cells.

As described herein, base editing is used to perform modifications of the kinome where amino acids that are necessary for catalytic activity of each kinase are selectively mutated. Other published technologies use base editing for therapeutic purposes (to edit mutant DNA that itself is causing a disease) or to model clinical variants found in patients. There are no technologies for high throughput modeling of endogenous enzyme inhibition (to mimic small molecule inhibition). The approach described herein utilizes pooled screens, providing an internal control to study enzyme inhibition and effects on cell growth (or other phenotypes). The advantage of the approach described herein is that it mimics enzyme inhibition (as a catalytic small molecule would) unlike knockdown/knockout approaches which remove the entire gene and do not recapitulate enzymatic inhibition.

Thus, possible applications for the invention include, but are not limited to, fast genetic screens to model effects of kinase inhibitor drugs (and inhibitors to other enzymes) in cells or in animal models.

Various other inventive aspects can be integrated or employed, as discussed infra.

While the invention has been particularly shown and described with reference to a preferred embodiment and various alternate embodiments, it will be understood by persons skilled in the relevant art that various changes in form and details can be made therein without departing from the spirit and scope of the invention.

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57 (b)(1), to relate to each and every individual publication, database entry (e.g. Genbank sequences or GeneID entries), patent application, or patent, each of which is clearly identified in compliance with 37 C.F.R. § 1.57 (b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1—Base Editing Enzyme Catalytic Activities in Cells

### Background and Objective

There is no tool to study the effects of or model endogenous catalytic inhibition of enzyme classes at scale across the human genome/proteome. Development of such a tool would allow for the precise functional understanding of the effects of enzyme catalytic inhibition in cells, mimicking the effects of small molecule inhibitors, in various diseases such as cancer. Such a tool would compress the interval between enzyme hit and lead discovery from decades to years by identifying the optimal mechanisms to inhibit enzymes, transforming drug developments and design of combination therapies.

Current methods of studying enzyme inhibition are inefficient and limited by technological constraints. Traditional CRISPR and RNA knockout (KO) and knockdown (KD) methods do not capture pharmacological intervention and are inefficient for performing knock-ins. Genetic KO screens are imperfect tools because absence of a protein rarely mimics inhibition of an intact protein. For example, for some genes, KO mutants are not lethal but catalytic inactivating mutants cause cell lethality, and vice versa, due to competing functions of domains outside the enzymatic domain and upregulation of genes that compensate for enzymatic loss. Pharmacologic screens are imperfect tools: many enzymes lack potent inhibitors, and existing inhibitors can be promiscuous where off-target effects may drive a phenotype. These issues compromise the utility of drug screens to discover new druggable enzyme targets. Nominating an enzyme target and then proving that catalytic inhibition causes cell lethality can take years and significant resources. A genetic tool to selectively disable enzyme catalysis while retaining physiological protein levels would accelerate our understanding of enzyme function in cells by mimicking the effects of enzyme inhibitors.

Existing methods to turn off enzyme catalysis suffer from lack of both scalability and endogenous perturbation. Overexpressing mutated genes can model enzymatic inhibition, but data interpretation is confounded by nonphysiologic levels of mutant protein expression. CRISPR knock-in mutagenesis at endogenous genomic loci is extremely inefficient, requiring isolation of single cells. Using knock-in to inhibit all enzymes in a particular is unfeasible, as it would require thousands of arrayed experiments which would take decades for a single lab to complete. In contrast, new techniques such as base editing can efficiently install point mutations in genes at physiologic levels. Other groups have proven the feasibility of base editing to scan and mutate throughout a gene's primary sequence using tiling approaches but no one has ever developed the tools to use base editing for precise, surgical inhibition of a large and diverse family of enzymes in a comprehensive manner.

This technology is a pooled base editing platform that targets the entire class of human kinase enzymes (the kinome) at physiologic levels in cells, and selectively inhibits these enzymes at the genomic level with precise mutations that better capture the mechanisms of pharmacological inhibition. Using kinase family-specific libraries, this platform uses base editing to perform precise DNA edits that effectively inhibit enzymatic activity once translated in a way that captures well-defined and FDA-approved pharmacological mechanisms. This platform can be used in therapeutic discovery to study the selective inhibition of kinases on cell growth in proliferation in various cancers for fast nomination of therapeutic targets.

### Description

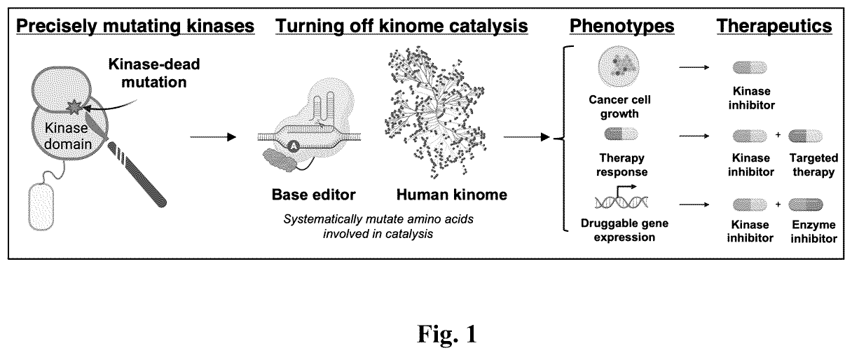

Compositions and methods described herein include cutting-edge base editing tools to precisely mutate amino acids necessary for enzyme catalysis in every enzyme member of the kinase class, and their use in analyzing how kinase catalysis regulates cancer cell function and identifying novel therapeutic targets to enable drug discovery (FIG. 1). Just as an engine is essential to keep a car running, catalysis is critical for enzyme function. If the engine is broken, the car is dead and will not run. Studying how a car performs in a complex system is best accomplished not by destroying the entire car and sorting through the rubble, but rather by altering specific parts, some of which are necessary for function (engine, tires) and others which are dispensable (color of the car). Analogously, for enzymes, KO and KD are crude tools that remove the entire gene, yielding insufficiently detailed or difficult to interpret information about enzyme cellular functions. Moreover, neither KO nor KD approaches necessarily translate to the effects of a small molecule inhibitor, and unsurprisingly have yielded few standard anticancer therapies. Therefore, studying how an enzyme functions in cells to enable drug discovery is best accomplished by altering its specific amino acids necessary for catalytic function, which will act as a surrogate for small molecule inhibition.

Current base editing tools cannot selectively turn off endogenous kinase catalytic activity in cells at scale. Described herein are compositions and methods to selectively mutate amino acids necessary for kinome catalysis to create a base editing platform, by 1) engineering an adenine base editor (ABE) that can bind to any gene sequence for site-specific editing; 2) identifying catalytically necessary amino acids for every human kinase; and 3) synthesizing a guide RNA (gRNA) library to edit catalytic amino acids and splice sites resulting in catalytic loss and whole gene loss:

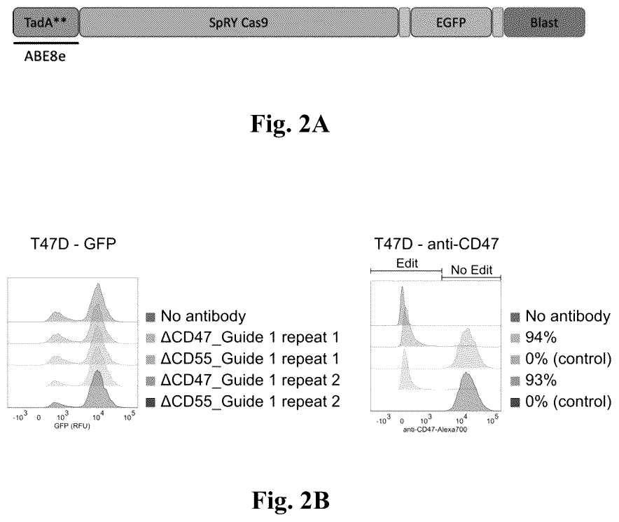

Adenine base editor: Base editing creates precise point mutations from C→T or A→G in genomic DNA. Base editors are comprised of fusions between a Cas9 nickase and a cytosine or adenine deaminase^{1,2}. Upon binding to the target locus, base pairing between the gRNA molecule and target DNA strand displaces a small segment of DNA, forming an editing window that can be modified by the deaminase. Compared with CRISPR knock-in, base editing is much more efficient; does not require single-cell isolation, which can introduce artifacts; and is well suited to functionally assess thousands of gene variants in a single experiment³⁻⁵. Here, adenine base editing is used, which allows for mutation of all possible codons of the amino acids responsible for kinase catalysis, unlike cytosine base editing, which could mutate only a subset. Base editors require a protospacer adjacent motif (PAM) site (usually NGG) at 13-19 base pairs downstream of the targeted base for DNA binding. This critical limitation often prevents base editors from introducing a desired point mutation. Therefore, a base editor containing 1) ABE8e, an adenine deaminase associated with one of the highest editing efficiencies (˜40-80% for most of the sites tested)⁶; and 2) SpRY-Cas9, a Cas9 ortholog with greater target site flexibility (NGN, NAN, and, to a lesser extent, NCN and NTN PAMs) that can bind to any sequence for site-specific editing⁷ was generated (FIG. 2A). This plasmid was transduced to create a variety of stable cell lines for the experiments. Base editing was confirmed in T47D estrogen receptor positive (ER+) breast cancer cells using a CD47 gRNA targeting its own splice site which causes functional knockout⁸ (FIG. 2B).

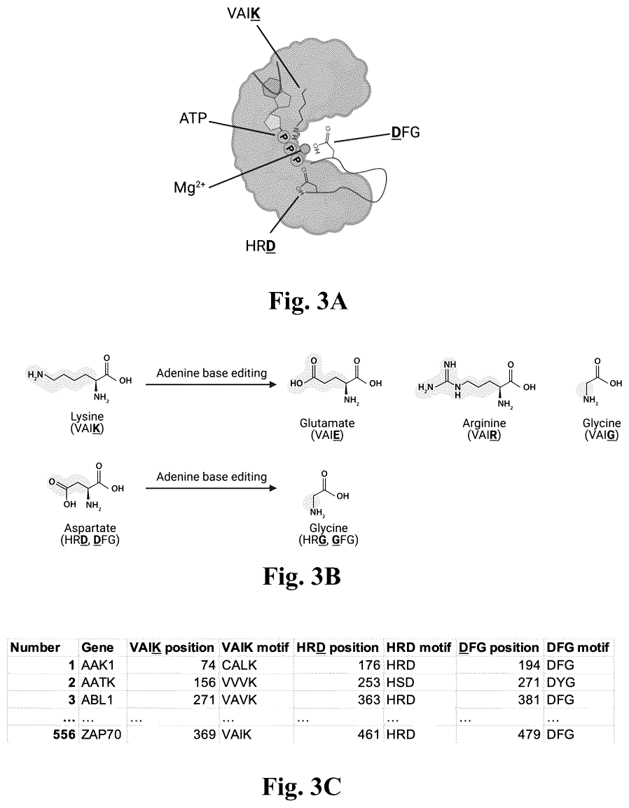

Enzyme protein engineering: First, catalytically necessary amino acids in every human kinase were identified. The protein kinase fold is well-conserved, and almost every kinase contains three invariant amino acids located in canonical sequence motifs: 1) VAIK, whose lysine (K) coordinates the phosphates in ATP; 2) HRD, whose aspartate (D) is the catalytic base for the kinase reaction; and 3) DFG, whose aspartate (D) binds to magnesium ions necessary for ATP coordination⁹ (FIG. 3A). Mutations at any of these sites abrogate kinase activity⁹⁻¹⁴ and are used to model catalytically dead kinases in proteins, cells, and mice. Specifically, adenine base editing would result in amino acid mutations of K→(E/R/G) and D→G, which invert charge (K→E), eliminate charge (K→G, D→G), or increase size (K→R) (FIG. 3B). These mutations kill kinase activity because amino acids with charge inversion or elimination cannot perform ATP transfer chemistry, and amino acids with increased size (even with identical charge as K→R) cannot be sterically accommodated in the active site to coordinate ATP^{9-11,14}. Each of these motifs were identified in every human and mouse kinase using bioinformatic analysis of sequence alignments and located their amino acid and genomic coordinates (FIG. 3C).

Kinome gRNA library design: gRNA molecules were designed to edit catalytic residues in every human kinase. Specifically, gRNA molecules were designed to edit catalytic residues, targeting at least one site in ˜97% of kinases and two or three sites in ˜91% of kinases. Adenine base editing cannot create stop codons², necessitating an alternative approach to model total gene loss, so splice donor sites are also edited which leads to effective KO⁸. The entire human and mouse libraries contain ˜6,000 gRNAs each, with 5 kinase-dead and 5 kinase KO gRNAs on average for each kinase. The library was cloned into a lentiviral vector with puromycin resistance. The library is an order of magnitude smaller than conventional whole-genome gRNA libraries (40,000-180,000 gRNAs), which enables rigorous analyses with multiple replicates.

Base editing kinase catalytic function in cells: After these technical developments and optimizations, a guide library targeting 22 kinases for base editing was selected (Table A).

TABLE A
Base editing pilot experiment gRNA library details.

| | | # of | | | # of |
|---|---|---|---|---|---|
| | # of | splice | # of | | essential |
| | essential | site | catalytic | # | genes |
| Family | genes | guides | guides | total | observed* |
| Kinases | 22 | 66 | 244 | 310 | 10 (45%) |

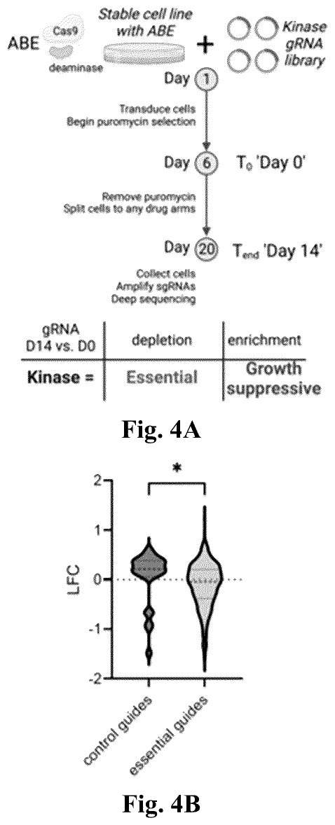

This library was used to perform a pilot base editing experiment in T47D estrogen receptor positive (ER+) breast cancer cells to determine which enzymes when catalytically inhibited cause ER+ breast cancer cell lethality (i.e. catalytic function is essential) or increased growth (i.e. catalytic function is growth suppressive) (FIG. 4A). Essential guides showed a greater log fold change (LFC) compared to control guides (FIG. 4B).

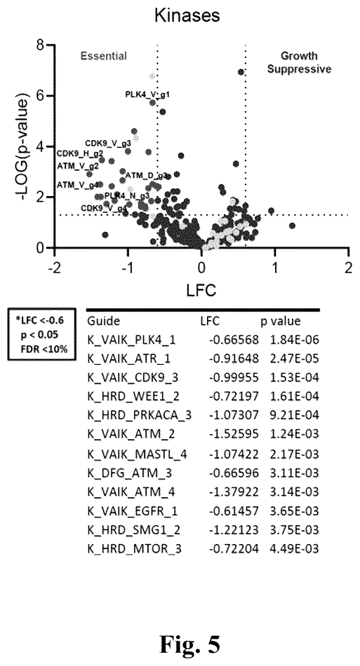

The kinase family is shown in a separate panel for clarity (FIG. 5).

Focusing on kinases (FIG. 5), the data showed that ATM, PLK4, and CDK9 kinase catalytic inhibition leads to triple-negative breast cancer (TNBC) cell lethality. The ATM phenotype is particularly striking since ATM genetic catalytic inhibition causes embryonic lethality in vivo^{15,16}, even though it is not an essential gene as shown by KO in vitro and in vivo¹⁵⁻¹⁷ and is a tumor suppressor in many cancer types. Importantly, each kinase gene candidate is represented by guides targeting multiple distinct catalytic sites which, as discussed above, are predicted to similarly kill kinase activity, showing the robustness of the general approach to create kinase dead mutants.

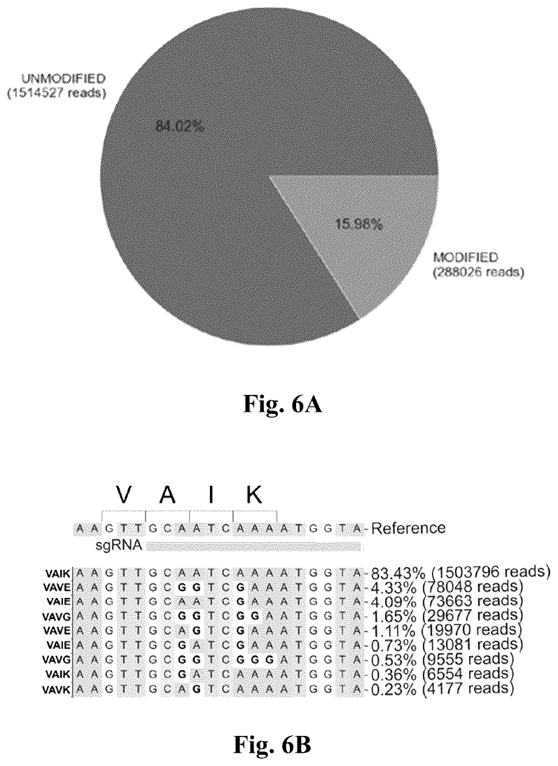

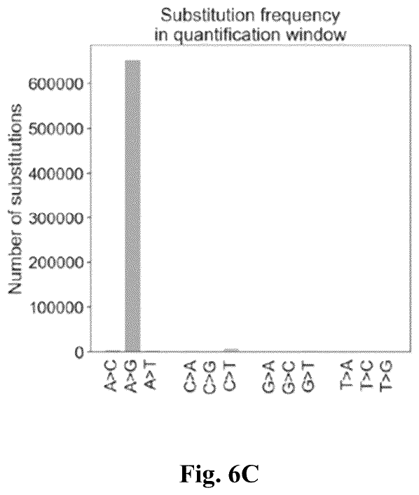

To confirm base editing of the catalytic site, the top kinase hit gRNA, K_VAIK_PLK4_1 (a gRNA editing the VAIK catalytic lysine in PLK4) was focused on. T47D cells were edited and it was found that 16% of sequences were modified (FIG. 6A), the vast majority of which showed edits at the catalytic lysine (FIG. 6B). Virtually all edits were A>G confirming ABE specificity (FIG. 6C).

This overall conceptual framework may be expanded in two large scale directions: 1) investigating enzyme inhibition in additional normal cell lines and cancer cell lines reflecting a wide variety of tumor types and histologies, and 2) analyzing a large number of functional readouts and phenotypes, including cell viability, cell viability in the setting of chemotherapy and targeted therapy treatment, gene expression, and cell surface expression of immune checkpoint markers (e.g. PD-L1).

### Variations

Certain edits (but not all, for the chosen enzymes) can be made at similar sites using a cytosine base editor but the amino acid chemistry is not as rigorous to confirm enzyme dead activity, as the resulting edits may still bind ATP or engage in ATP transfer. Importantly, the gRNA sequences themselves would remain identical even if a cytosine base editor were deployed. Prime editing can directly mutate amino acids to any other amino acid (such as alanine which would be ideal) but prime editing techniques currently suffer from extremely low efficiencies (<10%) so could not be deployed to inhibit a large family of enzymes.

### Summary

There is no available base editing approach or published gRNAs to specifically inhibit enzyme activity. Some groups have used base editing to model oncogenic mutations or perform mutational scanning using tiling based approaches^{3-5,18}.

Using traditionally CRISPR-Cas9 approaches that create double stranded breaks, the Vakoc group (Vakoc Human Kinase Domain-Focused CRISPR Knockout Library, Addgene). Has developed a lentiviral CRISPR knockout library that contains gRNAs that tiles across only the kinase domain of protein kinases. Targeting the functional domain of proteins enhances the severity of the negative selection screen. This is not base editing-based, and the approach described herein is more precise as it specifically targets enzyme activity.

In this invention, a set of guide RNAs for precise, surgical inhibition of kinase enzymes, mimicking the effects of small-molecule active-site inhibitors is disclosed. It represents an impactful advance, challenging traditional dogma of conventional gene KO studies because it will directly implicate catalytic mechanisms in driving a phenotype. This work will also directly compare for the first time the functional effects of enzyme catalytic inhibition and KO, enabling rational kinase targeting by the drug type that maximizes cell lethality: a catalytic inhibitor versus a protein degrader. The technical innovation described herein will enable drug discovery in a drastically shorter timespan (years versus decades), which will translate into practical innovation. Moreover, as all enzyme classes contain conserved catalytic amino acids whose mutation kills enzyme activity, approach described herein is scalable to inhibit any enzyme class for cancer eradication.

Identifying new enzyme targets will commercially transform oncology and other diseases with potential pharma, biotech, and basic science users/customers through 1) immediate clinical testing of existing enzyme active-site inhibitors as monotherapy or combination; 2) long-term drug development investments to develop more selective enzyme active-site inhibitors, monoclonal antibodies, and allosteric inhibitors; 3) material use of the gRNA sequences as therapeutic leads themselves for base editing therapies for germline genetic diseases or cancers caused by activating enzyme mutations, in a “one size fits all” manner killing enzyme activity rather than correcting individual mutations one at a time.

### Example 1

### REFERENCES

1. Komor, A. C., Kim, Y. B., Packer, M. S., Zuris, J. A. & Liu, D. R. Programmable editing of a target base in genomic DNA without double-stranded DNA cleavage. Nature 533, 420-424, doi:10.1038/nature17946 (2016).
2. Gaudelli, N. M. et al. Programmable base editing of A*T to G*C in genomic DNA without DNA cleavage. Nature 551, 464-471, doi:10.1038/nature24644 (2017).
3. Hanna, R. E. et al. Massively parallel assessment of human variants with base editor screens. Cell 184, 1064-1080 e1020, doi:10.1016/j.cell.2021.01.012 (2021).
4. Cuella-Martin, R. et al. Functional interrogation of DNA damage response variants with base editing screens. Cell 184, 1081-1097 e1019, doi:10.1016/j.cell.2021.01.041 (2021).
5. Sangree, A. K. et al. Benchmarking of SpCas9 variants enables deeper base editor screens of BRCA1 and BCL2. Nat Commun 13, 1318, doi:10.1038/s41467-022-28884-7 (2022).
6. Richter, M. F. et al. Phage-assisted evolution of an adenine base editor with improved Cas domain compatibility and activity. Nat Biotechnol 38, 883-891, doi:10.1038/s41587-020-0453-z (2020).
7. Walton, R. T., Christie, K. A., Whittaker, M. N. & Kleinstiver, B. P. Unconstrained genome targeting with near-PAMless engineered CRISPR-Cas9 variants. Science 368, 290-296, doi:10.1126/science.aba8853 (2020).
8. Kluesner, M. G. et al. CRISPR-Cas9 cytidine and adenosine base editing of splice-sites mediates highly-efficient disruption of proteins in primary and immortalized cells. Nat Commun 12, 2437, doi:10.1038/s41467-021-22009-2 (2021).
9. Huse, M. & Kuriyan, J. The conformational plasticity of protein kinases. Cell 109, 275-282, doi:10.1016/s0092-8674(02)00741-9 (2002).
10. Iyer, G. H., Moore, M. J. & Taylor, S. S. Consequences of lysine 72 mutation on the phosphorylation and activation state of cAMP-dependent kinase. J Biol Chem 280, 8800-8807, doi:10.1074/jbc.M407586200 (2005).
11. Gibbs, C. S. & Zoller, M. J. Rational scanning mutagenesis of a protein kinase identifies functional regions involved in catalysis and substrate interactions. J Biol Chem 266, 8923-8931 (1991).
12. Varjosalo, M. et al. Application of active and kinase-deficient kinome collection for identification of kinases regulating hedgehog signaling. Cell 133, 537-548, doi:10.1016/j.cell.2008.02.047 (2008).
13. Ahler, E. et al. A Combined Approach Reveals a Regulatory Mechanism Coupling Src's Kinase Activity, Localization, and Phosphotransferase-Independent Functions. Mol Cell 74, 393-408 e320, doi:10.1016/j.molcel.2019.02.003 (2019).
14. Kung, J. E. & Jura, N. Structural Basis for the Non-catalytic Functions of Protein Kinases. Structure 24, 7-24, doi:10.1016/j.str.2015.10.020 (2016).
15. Yamamoto, K. et al. Kinase-dead ATM protein causes genomic instability and early embryonic lethality in mice. J Cell Biol 198, 305-313, doi:10.1083/jcb.201204098 (2012).
16. Daniel, J. A. et al. Loss of ATM kinase activity leads to embryonic lethality in mice. J Cell Biol 198, 295-304, doi:10.1083/jcb.201204035 (2012).
17. Tsherniak, A. et al. Defining a Cancer Dependency Map. Cell 170, 564-576 e516, doi:10.1016/j.cell.2017.06.010 (2017).
18. Sanchez-Rivera, F. J. et al. Base editing sensor libraries for high-throughput engineering and functional analysis of cancer-associated single nucleotide variants. Nat Biotechnol 40, 862-873, doi:10.1038/s41587-021-01172-3 (2022).

### Example 2—Base-Editing the Cancer Kinome to Enable Drug Discovery

### Description

The human genome contains 556 kinase genes, commonly called the kinome^{1,2}. Kinases are enzymes that regulate nearly all aspects of cell signaling. Notably, abnormal cell signaling arising from many events including kinase dysregulation leads to excess cell proliferation and is a hallmark of cancer³. Kinases are among the most important and popular targets in precision oncology due to the case in assaying their activities in cells and designing small-molecule inhibitors. Although 85% of the kinome is dysregulated in cancer and other diseases⁴, however, most kinases are untapped as therapeutic targets—at present approved inhibitors target only 10% of the kinome⁵. The ideal kinase targets or kinase drug combinations for most cancers have not been established⁶. Determining the cellular consequences of turning off kinase enzymes has immense potential to transform our understanding of kinases and kinase inhibitors.

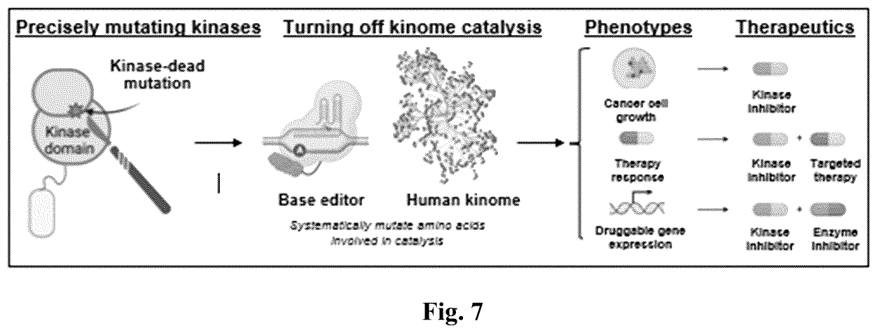

Compositions and methods described herein include cutting-edge base-editing tools to precisely mutate amino acids necessary for enzyme catalysis in every kinase across the kinome, and their use in analyzing how kinase catalysis regulates cancer cell function and identifying novel therapeutic targets to enable drug discovery (FIG. 7). Just as an engine is essential to keep a car running, enzyme catalysis is critical for kinase function. If the engine is broken, the car is dead and will not run. Studying how a car performs in a complex system is best accomplished not by removing the entire car, but rather by altering specific parts, some of which are necessary for function (engine, tires) and others which are dispensable (color of the car). Equally, for kinases, knock-out and knock-down are crude tools that remove the entire gene, yielding insufficiently detailed or difficult to interpret information about kinase cellular functions. Moreover, neither knock-out nor knock-down approaches necessarily translate to the effects of a small molecule inhibitor, and unsurprisingly have yielded few standard anticancer therapies. Therefore, studying how a kinase functions in cells to enable drug discovery is best accomplished by altering its specific amino acids necessary for catalytic function, which will act as a surrogate for small-molecule inhibition.

The base-editing approach to inactivate kinase catalysis as described herein solves two major issues that prevent existing mutagenesis methods from scaling across the kinome and performing mutations at physiologic levels. Classical site-directed mutagenesis can turn off kinase catalysis by overexpressing mutant kinase genes. This is scalable across the kinome but is non-physiologic as it artificially increases protein levels in cells⁷. Alternatively, gene knock-in can introduce point mutations at endogenous genomic loci to turn off catalysis kinase-by-kinase. However, knock-in is extremely inefficient, requiring single cell isolation to ensure mutation⁸, and so would take decades to scale across the kinome. In contrast, base editing can efficiently mutate hundreds of genes at physiologic levels in cells in a single pooled experiment⁹⁻¹³. Base-editing human kinases results in accelerated kinase target validation and drug discovery but requires a systematic high-throughput approach to edit catalytic activities endogenously and scale across the entire kinome.

Disclosed herein is a base editing platform to selectively mutate individual catalytic amino acids in 556 kinases at physiologic levels in cancer cells for pooled functional studies. A breast cancer model is used as a case study because multiple kinase inhibitors are standard therapies for breast cancer but combinatorial treatments are urgently needed. The base editing platform is used to interrogate how kinase activity regulates 1) cancer cell growth, 2) sensitivity to existing targeted therapies, and 3) druggable gene expression. Notably, this platform compresses the interval between kinome hit and lead discovery from decades to years by identifying the optimal mechanisms to inhibit kinases, transforming kinase drug developments and design of combination therapies.

### Background and Significance

Kinases in Normal and Cancer Cells: Kinases transfer phosphate from ATP to proteins, lipids, and other substrates¹⁴. Protein phosphorylation alters protein activity, stability, localization, and interactions, and lipid phosphorylation activates signaling and specifies membrane identity. Kinases function as molecular switches in virtually all cellular processes including cell growth, therapeutic responses, and gene expression. Despite its importance in biology, one third of the kinome has unknown function in cells¹⁵.

Kinase genes are frequently altered in cancer, where they play causal roles as oncogenes and tumor suppressors⁵. Advances in tumor genome sequencing have largely identified the kinase drivers of cancer. Kinase inhibitors including active-site inhibitors that mimic ATP binding are some of the most effective therapies in oncology⁵. Kinases also have noncatalytic functions that can be targeted by allosteric inhibitors or small-molecule protein degraders. Despite critical gains in targeting kinases in cancer, there have also been notable failures and challenges. Many cancers rewire cell signaling in specific contexts that vary by tissue type, genetic background, and drug treatment, resulting in alternative kinase dependencies⁵. For example, estrogen receptor-positive breast cancer is dependent on CDK4/6, PI3K, and mTOR kinases for cell growth. Combining inhibitors to any of these kinases with antiestrogen therapies improves patient survival versus antiestrogen therapy alone 16. Understanding kinase dependencies in cancer is critical for elucidating signaling pathways and discovering new kinases for therapeutic targeting; however, the functional diversity of kinases across the kinome complicates this effort. New approaches are needed that can handle this complexity at scale.

Models of Kinase Loss of Function and their Inadequacies for Target Validation: Unbiased whole-genome and kinome loss-of-function approaches such as RNA interference or clustered regularly interspaced short palindromic repeats (CRISPR)-Cas9 knock-out screens have established critical roles for kinases in cell growth, therapy response, and gene expression¹⁷⁻²³. Pharmacologic screens using small molecules are frequently used to inhibit kinase activity for drug discovery or repurposing²⁴. However, genetic and pharmacologic screens have resulted in few approved kinase inhibitors, and new unbiased tools are needed to understand kinase function.

Genetic knock-out screens are imperfect tools because absence of a protein rarely mimics inhibition of an intact protein²⁵⁻²⁷. For example, for some kinases, knock-out mutants are not lethal but catalytic inactivating mutants cause cell lethality, and vice versa^{27,28}, due to competing functions of domains outside the kinase domain²⁹ and upregulation of genes that compensate for kinase loss^{30,31}. Knock-outs simulate the effects of protein degrader molecules rather than catalytic inhibitors. Pharmacologic screens are imperfect tools: many kinases lack potent drug inhibitors, and existing kinase inhibitors can be promiscuous where off-target effects may drive a phenotype³². These issues compromise the utility of drug screens to discover new kinase targets. Nominating a kinase target and then proving that kinase catalytic inhibition causes cancer cell lethality can take years and significant resources. A high-throughput method to identify all kinases whose inhibition of catalytic activity causes cancer cell lethality overcomes this bottleneck.

Turning off Kinome Catalysis at Physiologic Levels: A genetic tool to selectively disable enzyme catalysis while retaining physiological protein levels accelerates our understanding of enzyme function in cells by mimicking the effects of enzyme inhibitors. Kinase activity is dependent on amino acids both in and distant from the active site. Certain amino acids in the kinase active site are conserved and shown to be necessary for catalysis in protein, cell, and mouse models consistently across the kinome. Site-directed mutagenesis of conserved active-site amino acids to create “kinase-dead” mutants is useful to test necessity of catalytic function^{7,33,34}. In contrast, amino acids distant from the active site are not conserved across the kinome and mutating them can have inconsistent effects on kinase activity, including inactivation, neutral effects, or activation. Mutating catalytic amino acids is superior to mutating distant amino acids to model loss of kinase catalysis at scale.

Existing methods to turn off kinase catalysis suffer from lack of both scalability and endogenous perturbation. Overexpressing mutated kinase genes can model kinome inhibition, but data interpretation is confounded by non-physiologic levels of mutant kinase protein expression⁷. CRISPR knock-in mutagenesis at endogenous genomic loci is extremely inefficient, requiring isolation of single cells. Using knock-in to inhibit all kinases in the kinome is unfeasible, as it would require thousands of arrayed experiments which would take decades for a single lab to complete. In contrast, techniques such as the base editing platform described herein can efficiently install point mutations in genes at physiologic levels^{9,10}. Other groups have proven the feasibility of base editing to scan and mutate throughout a gene's primary sequence using tiling approaches¹¹⁻¹³ but no one has ever deployed base editing for precise surgical inhibition of a large and diverse family of enzymes in a comprehensive manner.

Unlocking the Potential of the Kinome for Cancer Drug Targets: Modeling the catalytically dead kinome enables the systematic analysis of how kinases regulate cancer cell growth, response to targeted therapies, and druggable gene expression. Such an approach can be utilized to serve as a discovery platform for detailed mechanistic inquiry into the downstream signal transduction molecules and pathways that mediate these phenotypes. It also accelerates the nomination of kinase targets for pharmacologic inhibition in cancer, either as monotherapy or in combination with approved therapies, compressing a timeline of decades into a few years.

### Technology Development to Overcome Previous Limitations

Available genetic tools cannot selectively turn off endogenous kinase catalytic activity in cells at scale. Described herein is a base editing approach to selectively mutate amino acids necessary for kinome catalysis. To engineer such a approach, a suite of new tools was developed, including: 1) an adenine base editor that can bind to any genomic sequence to enable site-specific editing; 2) identification of catalytically necessary amino acids for every human kinase; 3) a guide RNA (gRNA) library to edit catalytic amino acids and splice sites resulting in catalytic loss and whole kinase gene loss, respectively; and 4) sophisticated proteomic strategies to prove on-target loss of kinase catalysis in cells.

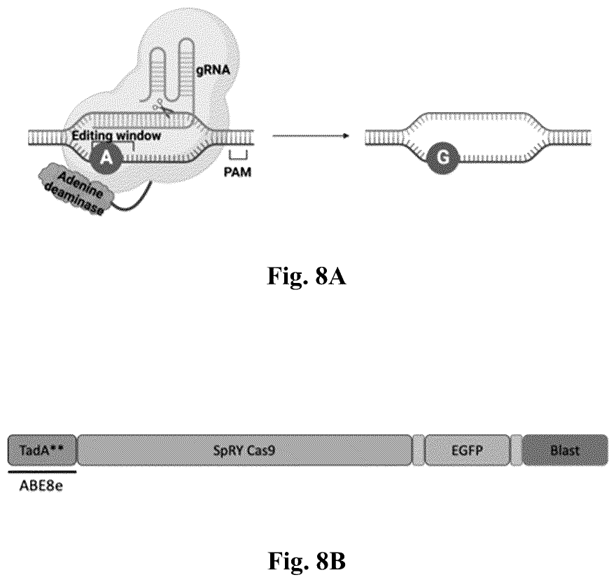

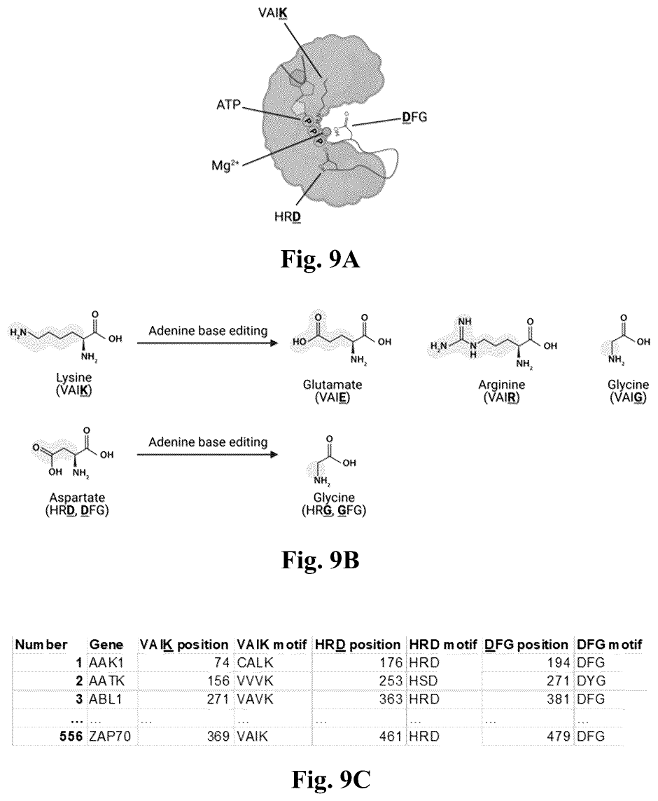

Adenine base editor: Base editing generates precise point mutations from C→T or A→G in genomic DNA. Base editors are comprised of fusions between a Cas9 nickase and a cytosine or adenine deaminase^{9,10}. Upon binding to the target locus, base pairing between the gRNA molecule and target DNA strand displaces a small segment of DNA, forming an editing window that can be modified by the deaminase (FIG. 8A). Compared with CRISPR knock-in, base editing is much more efficient; creates nearly 100% homozygous mutations in cells that undergo editing^{9,10}; does not require single-cell isolation, which can introduce artifacts; and is well suited to functionally assess thousands of gene variants in a single experiment¹¹⁻¹³. Adenine base editing is preferred for this purpose because it allows for mutation of all possible codons of the amino acids responsible for kinase catalysis (FIGS. 9A-9C), unlike cytosine base editing, which can mutate only a subset.

Base editors require a protospacer adjacent motif (PAM) site (usually NGG) at 13-19 base pairs downstream of the targeted base for DNA binding. This critical limitation often prevents base editors from introducing a desired point mutation. Therefore, a base editor containing 1) ABE8e, an adenine deaminase associated with one of the highest editing efficiencies (˜40-80% for most of the sites tested)³⁸; 2) SpRY-Cas9, a Cas9 ortholog with greater target site flexibility (NGN, NAN, and, to a lesser extent, NCN and NTN PAMs) that can bind to any sequence for site-specific editing³⁹; and 3) a GFP marker and blasticidin resistance cassette for flow cytometry and antibiotic selection was generated (FIG. 8B). A plasmid encoding the engineered base editor was transduced to create a variety of stable cell lines with high levels of base editor expression.

Kinase protein engineering: The protein kinase structural fold is well conserved throughout evolution, and almost every kinase contains three invariant amino acids located in canonical sequence motifs: 1) VAIK, whose lysine (K) coordinates the phosphates in ATP; 2) HRD, whose aspartate (D) is the catalytic base for the kinase reaction; and 3) DFG, whose aspartate (D) binds to magnesium ions necessary for ATP coordination³³ (FIG. 9A).

Mutations at any of these sites abrogate kinase activity^{7,25,33,34,40,41} and are frequently used to model catalytically dead kinases in protein, cell, and mouse models. Specifically, adenine base editing would result in amino acid mutations of K→(E/R/G) and D→G, which invert charge (K→E), eliminate charge (K→G, D→G), or increase size (K→R) (FIG. 9B). These mutations kill kinase activity because amino acids with charge inversion or elimination cannot perform ATP transfer chemistry, and amino acids with increased size (even with identical charge as with K→R) cannot be sterically accommodated in the active site to coordinate ATP^{25,33,34,40}. To generate the base editing platform, each of these motifs in every kinase in the human kinome were identified using bioinformatic analysis of sequence alignments, and their amino acid and genomic coordinates were located for base editing (FIG. 9C). The only kinases without these sites were known pseudokinases, genes with sequences similar to kinases but which lack kinase activity.

Kinome gRNA library design: gRNAs molecules were then designed to edit catalytic residues, targeting at least one site in ˜97% of kinases and two or three sites in ˜91% of kinases. Some sites are lost through either intrinsic lack of catalytic residues (pseudokinases¹) or the presence of termination sequences⁴². Adenine base editing cannot create stop codons¹⁰, necessitating an alternative approach to model gene loss, so splice donor sites editing, which leads to effective knock-out⁴³, is used for that purpose. The pooled design of the assay enables direct comparison between kinase-dead and knock-out conditions—and does so at the scale of the entire human kinome. For positive controls, splice sites in known essential genes are edited, as well as genes encoding cell-surface proteins to confirm editing by flow cytometry. For negative controls, nontargeting guides, empty-window guides targeting essential loci without adenines in the editing window¹², and guides targeting safe harbor gene loci that do not affect the cell⁴⁴ are used.

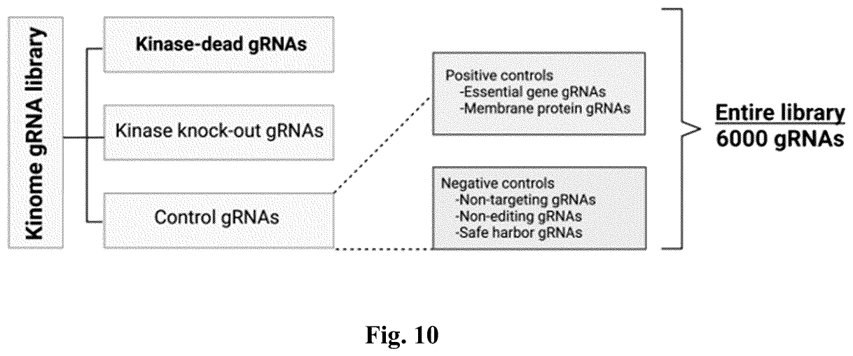

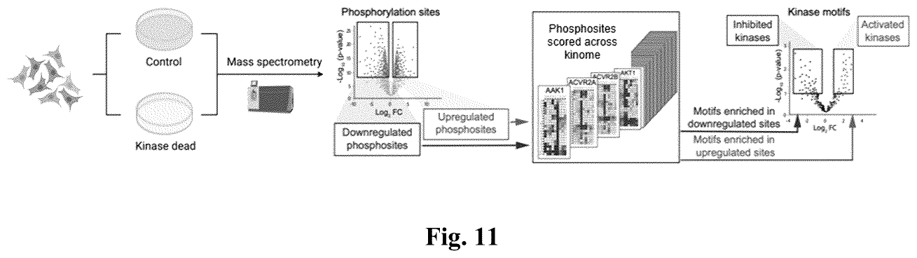

The entire library contains ˜6,000 gRNAs (FIG. 10), with 5 kinase-dead and 5 kinase knock-out gRNAs on average for each kinase. The library was cloned into a lentiviral vector with puromycin resistance. Notably, the library is an order of magnitude smaller than conventional whole-genome gRNA libraries (40,000-180,000 gRNAs), which enables rigorous analyses with multiple replicates.

### Experimental Strategy

Kinase Function in Cells: The base-editing platform may be utilized to answer three unsolved questions in how kinases control cancer cells: 1) which kinase activities are essential for cancer cell growth; 2) how kinase catalytic inhibition increases sensitivity to targeted therapies in breast cancer; and 3) how kinase catalytic inhibition alters druggable gene expression in breast cancer. Answering these questions determines the precise effects of inhibiting all human kinases on cancer phenotypes, at unprecedented scale and selectivity.

Which kinase activities are essential for cell growth? Gene knock-down/knock-out studies have determined the repertoire of kinases essential for cell growth, but provide little insight across the kinome into which parts of kinases, catalytic or noncatalytic, affect cell growth. Using the compositions and methods described herein, parallel mutagenesis of all kinases in the kinome in pooled cellular assays can be performed to identify those whose catalytic inhibition results in increased lethality. Splice site “knock-out guides” targeting all 556 kinases in the same library may also be included (B.3.c. gRNA library design). These “knock-out guides” serve for two important purposes: 1) to knock out essential kinases which will cause cell lethality, to use as positive controls for base editing; and 2) to directly compare the cellular consequences of kinase-dead and knock-out conditions at the scale of the entire human kinome. Various cell lines may be used, including HAP1 leukemia cells, which are commonly used for gene editing because they are haploid, removing any possibility of confounding heterozygous mutations. Other diverse cancer cell lines that are standard models in gene editing and express the adenine base editor described herein, including MDA-MB-231 (breast), A375 (skin), HT29 (colon), and A549 (lung), may also be used.

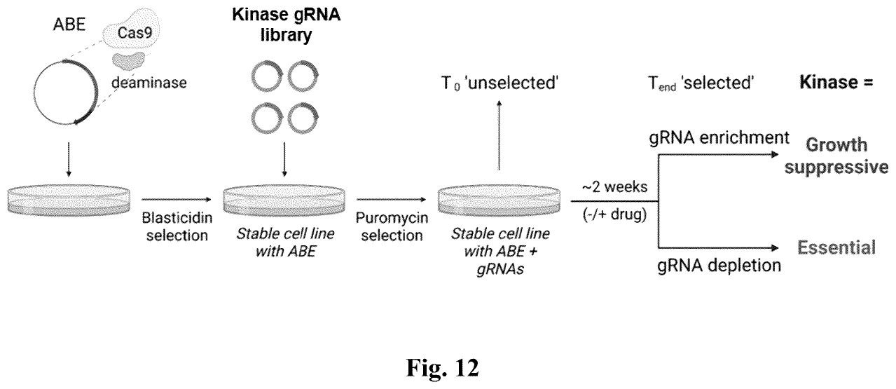

Stable cell lines bearing the adenine base editor are lentivirally transduced with the gRNA library and selected with puromycin for two (2) days. A sample is collected (T₀ “unselected”), cells are cultured for two (2) weeks for growth selection, and then another sample is collected (T_{end} “selected”) (FIG. 12). For both timepoints, genomic DNA is isolated and the integrated lentiviral cassette containing the gRNA is amplified and sequenced using next generation sequencing (NGS). Each screen is performed in at least three replicates to accurately measure enrichment/depletion of each gRNA.

For computational analysis, guide depletion (essential kinases) or enrichment (growth-suppressive kinases) for each kinase is ranked compared to unselected sample for each cell line using Robust Rank Aggregation⁴⁷. Guides targeting catalytic residues are grouped separately from guides targeting splice sites for each kinase to compare the growth effects of loss of catalytic activity with those of loss of the whole kinase. Of particular interest are 1) kinases causing discordant cell-growth phenotypes as kinase-dead mutants versus kinase knock-out mutants and 2) kinases causing differing phenotypes in different cell types. For validation, the top 5 kinases with the greatest positive and negative log fold changes in cell growth as kinase-dead versus kinase knock-out mutants are prioritized. To determine the mechanisms underlying the effects of kinase catalytic inhibition versus kinase loss on cell growth, isogenic kinase-dead and kinase knock-out stable cell lines are generated with the guides described herein. Adenine base editing and catalytic amino acid mutation is confirmed using NGS.

Additionally, 1) phosphorylation of known kinase substrates using Western blotting and 2) unbiased cellular phosphorylation changes using phosphoproteomics are measured (B.3.d. Phosphoproteomic motif validation). Accordingly, the base editing platform described herein may be used to analyze gene essentiality in cancer cells by precisely defining which kinases and which of their catalytic and noncatalytic functions affect cell growth.

How does kinase inhibition improve response to targeted therapies? Breast cancer is the most common cancer in women worldwide⁴⁸. The most common subtype of breast cancer is estrogen receptor-positive breast cancer (ER+ BC), which overexpresses the transcription factor ER and is dependent on estrogen. Anti-estrogen therapies such as the ER degrader fulvestrant are standard therapies that improve overall survival in patients with ER+ BC. Kinase dysregulation is a common feature of ER+ BC, and inhibitors of the kinases CDK4/6, mTOR, and PI3K increase the efficacy of fulvestrant and are approved therapies in ER+ BC¹⁶. Invariably, patients with metastatic ER+ BC will fail to respond or develop drug resistance, necessitating new tools for kinase target discovery. Knock-out screens have been performed in ER+ BC⁴⁹ but have largely not resulted in development of new drugs.

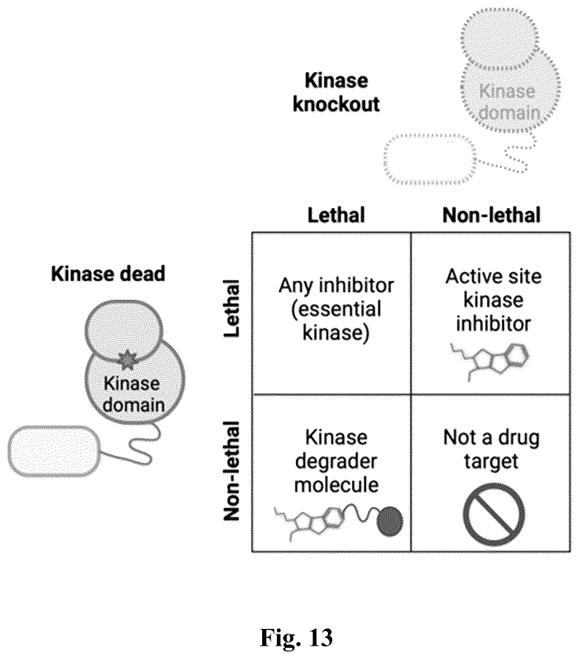

Editing the kinome to identify new targets whose catalytic inhibition increases breast cancer lethality is transformative for oncology because it enables 1) immediate clinical testing of existing kinase active-site inhibitors and 2) long-term investments to develop more selective kinase active-site inhibitors, monoclonal antibodies, and allosteric inhibitors. Kinase knock-out mimics the effects of a protein degrader rather than an active-site inhibitor (FIG. 13). Moreover, identifying kinases whose catalytic inhibition has no effect but whose knock-out increases cell lethality (borrowing the car analogy: turning off the engine is not enough; the car must be destroyed) will provide a rationale to test and develop targeted protein degraders for new target kinases (FIG. 13). Additionally, inhibition of many kinases alone without inhibiting ER is clinically ineffective in ER+ BC, pointing to the necessity of scalable tools to determine new combination therapies targeting kinases and ER.

Accordingly, the base-editing platform may be used to identify new kinase therapeutic vulnerabilities in ER+ BC in combination with fulvestrant. Stable MCF7 and T47D ER+ BC cell lines expressing the adenine base editor described herein were generated. These cells are standard models for ER+ BC and are sensitive to fulvestrant, as are patients. Using these cells, a negative selection with fulvestrant concentrations that kill 20% of cells is applied (FIG. 12). The following results are prioritized: 1) kinases that increase fulvestrant lethality when kinase-dead but not on knock-out as new targets for active-site kinase inhibitors (FIG. 13, upper right corner) and 2) kinases that increase fulvestrant lethality on knock-out but not when kinase-dead as new targets for kinase degraders⁵⁰ (FIG. 13, lower left corner). For top hits, isogenic kinase-dead and kinase knock-out cancer cell lines are created. Changes in cell proliferation and tumor formation (e.g., by injecting these cells into mice to create xenografts) on exposure to fulvestrant combined with kinase active-site inhibitors and kinase degraders⁵¹ are measured. Also, effects on ER-dependent transcription are measured using quantitative reverse transcription PCR. In parallel, positive selection experiments are performed to identify the kinases whose catalytic inhibition and knock-out cause fulvestrant resistance. This approach may be expanded to test other ER+ BC-targeted therapies including CDK4/6 and PI3K inhibitors. Together, these approaches will multiply the number of new kinase targets for combination therapies with kinase inhibitors and fulvestrant in ER+ BC.

How does kinase inhibition regulate druggable gene expression? The first two questions focus on cancer cell growth, but kinase inhibition can lead to diverse genetic changes that do not affect cell growth but might nonetheless be targeted with combination therapies. For example, PI3K inhibition in ER+ BC leads to increased ER-dependent transcription and combining PI3K and ER inhibitors is more effective than single therapies in breast cancer patients⁵²⁻⁵⁴. Recent advances in molecular phenotyping allow for single cell readouts of transcription that, when coupled with CRISPR genome editing, can refine our understanding of how genetic perturbations affect gene expression and cell state^{55,56}. Thus far, these large-scale approaches have used single-cell readouts for gene knock-out and transcriptional regulations only. Growth factors can induce a diverse range of gene expression by activating receptor kinases that turn on and off a multitude of downstream kinases and signaling pathways. Given this complexity, the significance of kinase catalysis across multiple signaling pathways on gene expression is poorly understood⁵⁷.

Accordingly, the compositions and methods described herein may be used to determine how kinase catalytic inhibition alters druggable gene expression by performing single-cell RNA sequencing on base-edited cells to nominate new kinase-kinase and kinase-phosphatase pairs for combination inhibition.=ER+ BC, which is a transcriptionally driven cancer and is primarily treated using combination therapies instead of single drugs, is a case example used to analyze kinase-druggable gene crosstalk mechanisms.

Single-cell RNA sequencing on kinase base-edited MCF7 cells using Expanded CRISPR-compatible CITE-seq (ECCITE-seq)⁵⁸ is performed. ECCITE-seq directly captures gRNA sequences using a custom reverse transcriptase primer for single-cell sequencing. Barcoded gel beads and reverse transcription reagents are combined with single-cell suspensions using microfluidics and partitioned into droplet emulsions. Cells are lysed, and mRNA transcripts are captured, reverse-transcribed, barcoded, and amplified into NGS libraries on a 10× Genomics instrument. 300,000 cells (3,000 gRNAs in the kinase-dead sublibrary at 100 cells per guide) are sequenced. To decrease the per-cell cost, ECCITE-seq's capabilities for cell overloading, sample multiplexing, and doublet cell detection (“Cell Hashing”) and deconvolution during sample preparation and data analysis⁵⁸ may be utilized.

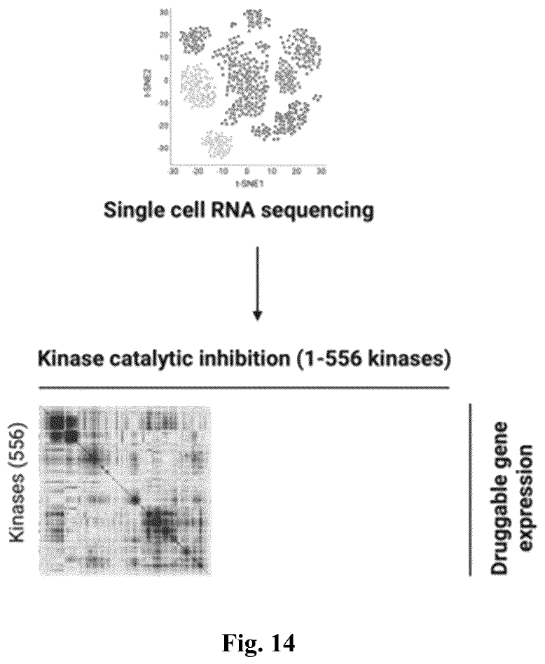

Data is visualized using single-cell dimensionality reduction (e.g., UMAP, t-SNE) to cluster transcriptionally similar cells and focus the analysis on subpopulations that are phenotypically distinct from wildtype. Transcriptional phenotypes are analyzed to interrogate how kinases catalytically regulate gene expression of kinases themselves and of phosphatases, a family of 198 druggable enzymes that remove phosphates from substrates^{60,61} and oppose the effects of kinases (FIG. 14).

This analysis delineates a kinase-druggable gene interdependency network and raise numerous hypotheses for kinase cross-talk mechanisms and new combination therapies. These hypotheses are validated using quantitative PCR and RNA sequencing of individual kinase base-edited cell lines. Combination drugging strategies targeting interdependent kinase-kinase and kinase-phosphatase pairs may be tested and cell lethalities may be compared by single-gene versus double-gene inhibition. Target gene expression in kinase-dead cells and in cells treated with small-molecule kinase inhibitors may be compared to determine on-and off-target changes. This approach is scalable to investigate the effects of kinase inhibition on the expression of other druggable genes (e.g., nuclear hormone receptors, immune checkpoints).

### Potential Alternative Approaches

Although base editing is less efficient than CRISPR-Cas9 nuclease editing (40-80% versus >90%), base-editing screens have nonetheless yielded many biologically and therapeutically important findings¹¹⁻¹³. To maximize sensitivity (i.e., increase true positives and decrease false negatives), each kinase-dead guide for a particular kinase for analysis may be binned together, and any kinase with at least one scoring guide (e.g., call as a positive hit a kinase where HRD guide scores but VAIK and DFG guides do not) may be set as a true positive threshold. If there are too few hits due to low base-editing efficiencies, it is possible to 1) increase the number of kinase-dead guides, targeting other conserved catalytic amino acids including the asparagine +5 after HRD⁶² and the glutamate +7 after VAIK62 (˜10 total guides per kinase), and 2) increase the number of cells in all pooled experiments by 5-fold to ensure a sufficient absolute number of base-edited cells for analysis.

### Innovation

Conceptual Innovation: A Genetic Approach to Enzyme Inhibition: A novel base editing platform for precise, surgical inhibition of a large, diverse family of enzymes in a comprehensive fashion. This platform will inhibit every kinase in the human genome, mimicking the effects of small-molecule active-site inhibitors. It represents an impactful advance from gene knock-out studies, which form the backbone of most knowledge of human kinase function, because it directly implicates catalytic mechanisms in driving a phenotype. This platform also allows for the direct comparison of the functional effects of kinase catalytic inhibition and knock-out for the first time, enabling rational kinase targeting by the drug type that maximizes cancer cell lethality: a catalytic inhibitor versus a protein degrader.

Technical Innovation: A Base-Editing Platform to Rapidly Disable Kinase Activity at Scale in Cells: The methods and compositions described herein leverage base editing to perform site-directed mutagenesis at scale to kill kinase activity, which contrasts with other uses of base editing to model oncogenic mutations or perform mutational scanning^{11-13,63}. This is the first single-cell readout of a high-throughput base-editor screening approach, enabling new kinase-druggable enzyme combination therapies.

Material Innovation: A gRNA library targeting 556 kinases at catalytic residues and a novel base editor with high editing efficiency and a liberalized PAM requirement. Moreover, as base editing is being investigated in vivo as a disease treatment⁶⁴, these gRNA sequences may be therapeutic utilized themselves for germline genetic diseases or cancers caused by activating kinase mutations.

### Example 2

### REFERENCES

1. Manning, G et al. Science 298, 1912 (2002).
2. Moret, N et al. bioRxiv, 2020.04.02.022277 (2021).
3. Hanahan, D et al. Cell 100, 57 (2000).
4. Wilson, LJ et al. Cancer Res 78, 15 (2018).
5. Ferguson, FM et al. Nat Rev Drug Discov 17, 353 (2018).
6. Essegian, D et al. Cell Rep Med 1, 100128 (2020).
7. Varjosalo, M et al. Cell 133, 537 (2008).
8. Ran, FA et al. Nat Protoc 8, 2281 (2013).
9. Komor, AC et al. Nature 533, 420 (2016).
10. Gaudelli, NM et al. Nature 551, 464 (2017).
11. Hanna, RE et al. Cell 184, 1064 (2021).
12. Cuella-Martin, R et al. Cell 184, 1081 (2021).
13. Sangree, AK et al. Nat Commun 13, 1318 (2022).
14. Hunter, T. Cell 100, 113 (2000).
15. Finan, C et al. Sci Transl Med 9 (2017).
16. Burstein, HJ. N Engl J Med 383, 2557 (2020).
17. Grueneberg, DA et al. PNAS 105, 16472 (2008).
18. Baldwin, A et al. PNAS 105, 16478 (2008).
19. Bommi-Reddy, A et al. PNAS 105, 16484 (2008).
20. Grueneberg, DA et al. PNAS 105, 16490 (2008).
21. Baldwin, A et al. PNAS 107, 12463 (2010).
22. Tsherniak, A et al. Cell 170, 564 (2017).
23. Behan, FM et al. Nature 568, 511 (2019).
24. Letai, A et al. Cancer Cell 40, 26 (2022).
25. Kung, JE et al. Structure 24, 7 (2016).
26. Knight, ZA et al. Cell 128, 425 (2007).
27. Weiss, WA et al. Nat Chem Biol 3, 739 (2007).
28. Newton, K. Trends Cell Biol 25, 347 (2015).
29. Mace, PD et al. J Biol Chem 296, 100705 (2021).
30. El-Brolosy, MA et al. Nature 568, 193 (2019).
31. El-Brolosy, MA et al. PLoS Genet 13, e1006780 (2017).
32. Knight, ZA et al. Nat Rev Cancer 10, 130 (2010).
33. Huse, M et al. Cell 109, 275 (2002).
34. Gibbs, CS et al. J Biol Chem 266, 8923 (1991).
35. Bailey, MH et al. Cell 173, 371 (2018).
36. Vasan, N et al. Science 366, 714 (2019).
37. Vasan, N et al. Nat Rev Clin Oncol 19, 471 (2022).
38. Richter, MF et al. Nat Biotechnol 38, 883 (2020).
39. Walton, RT et al. Science 368, 290 (2020).
40. Iyer, GH et al. J Biol Chem 280, 8800 (2005).
41. Ahler, E et al. Mol Cell 74, 393 (2019).
42. Gao, Z et al. Mol Ther Nucleic Acids 10, 36 (2018).
43. Kluesner, MG et al. Nat Commun 12, 2437 (2021).
44. Sadelain, M et al. Nat Rev Cancer 12, 51 (2011).
45. Needham, EJ et al. Sci Signal 12 (2019).
46. Johnson, JL et al. bioRxiv, 2022.05.22.492882 (2022).
47. Li, W et al. Genome Biol 15, 554 (2014).
48. Waks, AG et al. JAMA 321, 288 (2019).
49. Xiao, T et al. PNAS 115, 7869 (2018).
50. Bekes, M et al. Nat Rev Drug Discov 21, 181 (2022).
51. Donovan, KA et al. Cell 183, 1714 (2020).
52. Bosch, A et al. Sci Transl Med 7, 283ra51 (2015).
53. Toska, E et al. Science 355, 1324 (2017).
54. Andre, F et al. N Engl J Med 380, 1929 (2019).
55. Camp, JG et al. Science 365, 1401 (2019).
56. Stuart, T et al. Nat Rev Genet 20, 257 (2019).
57. Hill, CS et al. Sci STKE 1999, PE1 (1999).
58. Mimitou, EP et al. Nat Methods 16, 409 (2019).
59. Replogle, JM et al. Cell (2022).
60. Chen, MJ et al. Sci Signal 10 (2017).
61. Oprea, TI et al. Nat Rev Drug Discov 17, 317 (2018).
62. Kanev, GK et al. Trends Pharmacol Sci 40, 818 (2019).
63. Sanchez-Rivera, FJ et al. Nat Biotechnol 40, 862 (2022).
64. Newby, GA et al. Mol Ther 29, 3107 (2021).
65. Vasan, N et al. PNAS 107, 14176 (2010).
66. Vasan, N et al. Oncologist 19, 453 (2014).

### Example 3—Base-Editing Kinase Screen

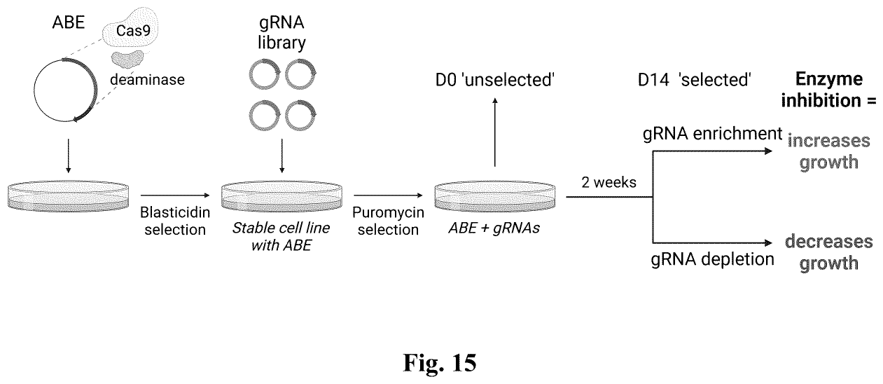

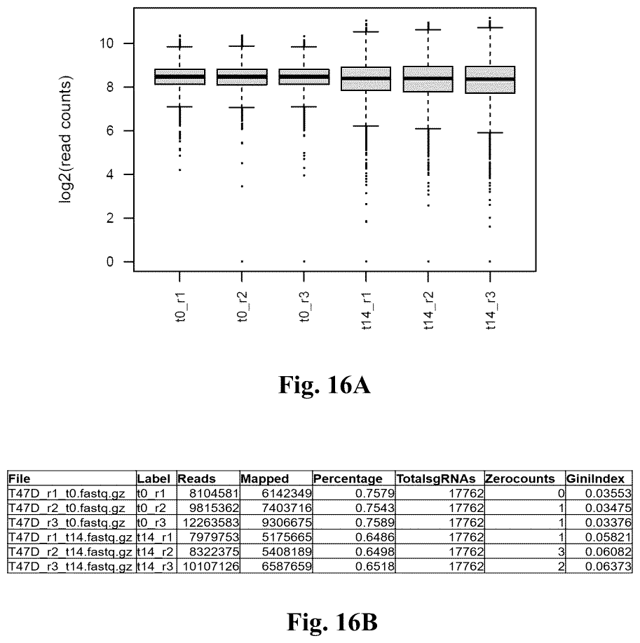

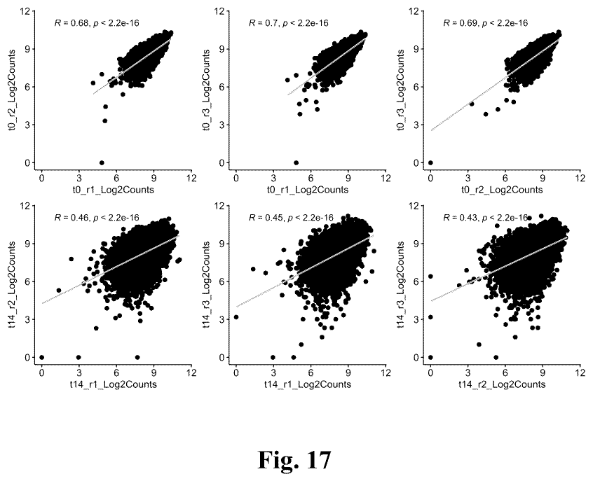

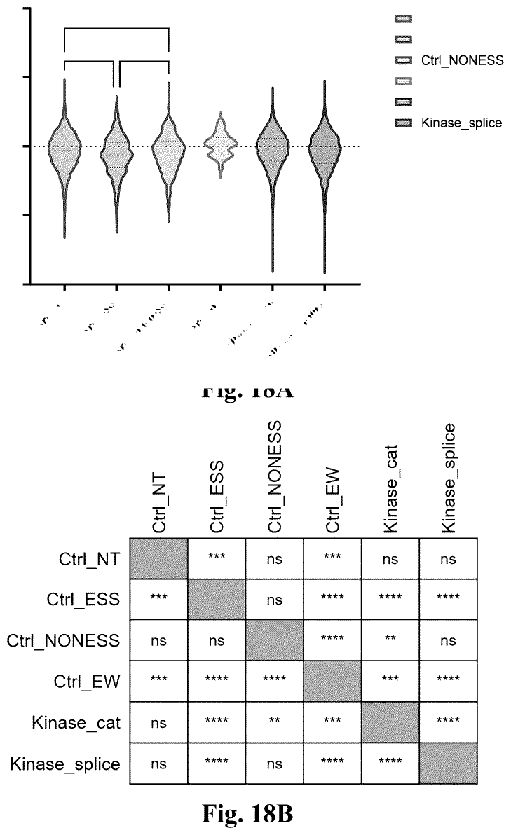

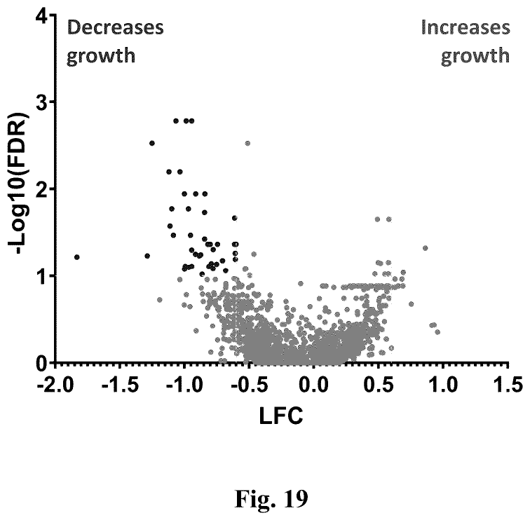

Results from a base editing kinase screen are shown in FIGS. 15-19. The top 50 sites decreasing the growth of T47D cells from MAGeCK RRA analysis are listed below.

TABLE B
Top 50 sites decreasing the growth of T47D cells.

| | | neg. | neg. p. | neg. | neg. |
|---|---|---|---|---|---|
| id | num | score | value | fdr | rank |
| RIPK2_VAIK | 18 | 8.2863E−08 | 1.9225E−06 | 0.00165 | 1 |
| RYK_DFG | 15 | 1.178E−07 | 5.7676E−06 | 0.00297 | 2 |
| MAP3K9_HRDplus5 | 18 | 1.4108E−07 | 1.9225E−06 | 0.00165 | 3 |
| MAP2K2_VAIK | 18 | 1.753E−07 | 1.9225E−06 | 0.00165 | 4 |
| Ctrl_ESS_PHB2 | 30 | 4.9189E−07 | 5.7676E−06 | 0.00297 | 5 |
| PIK3C3_HRD | 15 | 1.4304E−06 | 0.000017303 | 0.006365 | 6 |
| KSR1_splice | 30 | 2.5588E−06 | 0.000017303 | 0.006365 | 7 |
| ADCK1_VAIK | 18 | 3.0681E−06 | 0.000040373 | 0.011386 | 8 |
| ERN1_VAIK | 18 | 3.4636E−06 | 0.000044218 | 0.011386 | 9 |
| PRKDC_VAIK | 18 | 3.8268E−06 | 0.000044218 | 0.011386 | 10 |
| ITPKC_HRD | 18 | 7.4574E−06 | 0.000078823 | 0.016914 | 11 |
| CDK16_VAIK | 18 | 7.4773E−06 | 0.000078823 | 0.016914 | 12 |
| Ctrl_NONESS_BPIFB3 | 30 | 7.8169E−06 | 0.00011727 | 0.02157 | 13 |
| MAP3K7_HRD | 15 | 8.2868E−06 | 0.000094204 | 0.01866 | 14 |
| PRAG1_VAIK | 21 | 0.00001513 | 0.00022494 | 0.034071 | 15 |
| CDK5_HRD | 12 | 0.000016782 | 0.00015572 | 0.026733 | 16 |
| OBSCN_KD2_VAIK | 18 | 0.000022712 | 0.00026339 | 0.037679 | 17 |
| SPEG_splice | 30 | 0.000023881 | 0.00034029 | 0.043366 | 18 |
| BUB1B_HRD | 15 | 0.00002453 | 0.00022494 | 0.034071 | 19 |
| ACVRL1_splice | 30 | 0.000026413 | 0.00035951 | 0.043366 | 20 |
| TRIB3_splice | 30 | 0.000031996 | 0.00041334 | 0.043366 | 21 |
| RPS6KL1_VAIK | 18 | 0.000032379 | 0.0003672 | 0.043366 | 22 |
| Ctrl_ESS_PSMB4 | 30 | 0.000032551 | 0.00042103 | 0.043366 | 23 |
| TESK1_VAIK | 18 | 0.000036408 | 0.00039027 | 0.043366 | 24 |
| TRIB1_VAIK | 18 | 0.000037642 | 0.00040181 | 0.043366 | 25 |
| Ctrl_ESS_PUF60 | 30 | 0.000039149 | 0.00050178 | 0.049695 | 26 |
| Ctrl_NONESS_CASP2 | 30 | 0.00004605 | 0.0005979 | 0.054986 | 27 |
| BRSK1_HRD | 15 | 0.000047005 | 0.00052869 | 0.050422 | 28 |
| DCLK1_HRDplus5 | 18 | 0.000056836 | 0.00063251 | 0.056163 | 29 |
| TRIB3_VAIK | 18 | 0.000058317 | 0.00065558 | 0.056271 | 30 |
| MAPK15_HRDplus5 | 18 | 0.000061806 | 0.00069019 | 0.05733 | 31 |
| CDK9_HRD | 15 | 0.000073662 | 0.00075171 | 0.058956 | 32 |
| AATK_HRD | 15 | 0.00008491 | 0.00087859 | 0.064639 | 33 |
| RPS6KA5_KD2_HRD | 12 | 0.000085066 | 0.00075555 | 0.058956 | 34 |
| EIF2AK3_VAIK | 18 | 0.000088364 | 0.0009478 | 0.066765 | 35 |
| PRKCB_splice | 24 | 0.000093357 | 0.001067 | 0.072303 | 36 |
| MAP2K3_HRD | 15 | 0.000098741 | 0.00095934 | 0.066765 | 37 |
| PRKCH_VAIK | 18 | 0.00010627 | 0.0011131 | 0.073496 | 38 |
| MAPK14_VAIK | 18 | 0.00011857 | 0.00124 | 0.07788 | 39 |
| PIM3_splice | 30 | 0.00012562 | 0.001563 | 0.083849 | 40 |
| ADCK5_DFG | 15 | 0.00012669 | 0.00124 | 0.07788 | 41 |
| ALPK2_HRD | 15 | 0.00012938 | 0.0012746 | 0.078147 | 42 |
| TAOK2_HRD | 15 | 0.00013572 | 0.0013323 | 0.079784 | 43 |
| Ctrl_ESS_ATP6V0C | 18 | 0.00014074 | 0.001463 | 0.082759 | 44 |
| MAP3K3_HRD | 15 | 0.00014466 | 0.0014323 | 0.082759 | 45 |
| AXL_DFG | 15 | 0.00014855 | 0.0014784 | 0.082759 | 46 |
| WEE1_DFG | 15 | 0.0001547 | 0.0015246 | 0.083526 | 47 |
| Ctrl_NONESS_PRSS54 | 27 | 0.0001586 | 0.0018975 | 0.095807 | 48 |
| Ctrl_ESS_RPL4 | 30 | 0.00016228 | 0.0019937 | 0.098724 | 49 |
| MAP2K1_HRD | 15 | 0.00016703 | 0.0016591 | 0.087189 | 50 |

| | neg. | neg. | pos. | pos. p. | pos. |
|---|---|---|---|---|---|
| id | goodsgrna | lfc | score | value | fdr |
| RIPK2_VAIK | 17 | −1.0661 | 0.996 | 0.998 | 0.999998 |
| RYK_DFG | 13 | −1.2509 | 0.96225 | 0.98593 | 0.999998 |
| MAP3K9_HRDplus5 | 17 | −0.98684 | 0.99982 | 0.99984 | 0.999998 |
| MAP2K2_VAIK | 17 | −0.94366 | 0.92174 | 0.98052 | 0.999998 |
| Ctrl_ESS_PHB2 | 18 | −0.51128 | 0.46555 | 0.82597 | 0.999998 |
| PIK3C3_HRD | 12 | −1.1199 | 0.99962 | 0.99965 | 0.999998 |
| KSR1_splice | 20 | −1.035 | 0.34674 | 0.73382 | 0.999998 |
| ADCK1_VAIK | 13 | −1.0004 | 0.21357 | 0.52821 | 0.999998 |
| ERN1_VAIK | 15 | −0.91263 | 0.99976 | 0.99978 | 0.999998 |
| PRKDC_VAIK | 11 | −0.84195 | 0.79135 | 0.9432 | 0.999998 |
| ITPKC_HRD | 14 | −0.96916 | 0.99999 | 0.99998 | 0.999998 |
| CDK16_VAIK | 15 | −1.0981 | 0.8767 | 0.96895 | 0.999998 |
| Ctrl_NONESS_BPIFB3 | 23 | −0.61231 | 0.9756 | 0.99773 | 0.999998 |
| MAP3K7_HRD | 14 | −0.84379 | 0.99968 | 0.9997 | 0.999998 |
| PRAG1_VAIK | 17 | −0.9534 | 0.50171 | 0.81666 | 0.999998 |
| CDK5_HRD | 10 | −1.1121 | 0.775 | 0.90926 | 0.999998 |
| OBSCN_KD2_VAIK | 14 | −0.84417 | 0.51618 | 0.8119 | 0.999998 |
| SPEG_splice | 23 | −0.61456 | 0.93097 | 0.99228 | 0.999998 |
| BUB1B_HRD | 10 | −1.0849 | 0.825 | 0.94357 | 0.999998 |
| ACVRL1_splice | 21 | −0.80064 | 0.9955 | 0.99964 | 0.999998 |
| TRIB3_splice | 21 | −0.74447 | 0.30155 | 0.68946 | 0.999998 |
| RPS6KL1_VAIK | 12 | −0.81877 | 0.70526 | 0.90964 | 0.999998 |
| Ctrl_ESS_PSMB4 | 19 | −0.60101 | 0.70112 | 0.9392 | 0.999998 |
| TESK1_VAIK | 12 | −0.81354 | 0.52848 | 0.82025 | 0.999998 |
| TRIB1_VAIK | 14 | −0.61135 | 0.98724 | 0.99563 | 0.999998 |
| Ctrl_ESS_PUF60 | 21 | −0.77781 | 0.99084 | 0.99922 | 0.999998 |
| Ctrl_NONESS_CASP2 | 20 | −0.60753 | 0.57108 | 0.88669 | 0.999998 |
| BRSK1_HRD | 8 | −0.9436 | 0.050698 | 0.19441 | 0.804106 |
| DCLK1_HRDplus5 | 11 | −0.46293 | 0.987 | 0.99554 | 0.999998 |
| TRIB3_VAIK | 14 | −0.91471 | 0.99564 | 0.99794 | 0.999998 |
| MAPK15_HRDplus5 | 14 | −0.87391 | 0.16453 | 0.44913 | 0.975047 |
| CDK9_HRD | 13 | −0.88412 | 0.42777 | 0.72858 | 0.999998 |
| AATK_HRD | 10 | −0.60606 | 0.86571 | 0.95623 | 0.999998 |
| RPS6KA5_KD2_HRD | 9 | −1.2871 | 0.90104 | 0.95637 | 0.999998 |
| EIF2AK3_VAIK | 13 | −0.70741 | 0.73449 | 0.92215 | 0.999998 |
| PRKCB_splice | 17 | −0.79291 | 0.95256 | 0.99307 | 0.999998 |
| MAP2K3_HRD | 12 | −0.70558 | 0.98881 | 0.99395 | 0.999998 |
| PRKCH_VAIK | 14 | −0.75152 | 0.70355 | 0.90886 | 0.999998 |
| MAPK14_VAIK | 13 | −0.99297 | 0.47642 | 0.78495 | 0.999998 |
| PIM3_splice | 22 | −0.53147 | 0.99789 | 0.99979 | 0.999998 |
| ADCK5_DFG | 11 | −0.94426 | 0.99717 | 0.99726 | 0.999998 |
| ALPK2_HRD | 13 | −0.8112 | 0.94894 | 0.9822 | 0.999998 |
| TAOK2_HRD | 12 | −0.96455 | 0.95888 | 0.98479 | 0.999998 |
| Ctrl_ESS_ATP6V0C | 14 | −0.53237 | 0.87679 | 0.96898 | 0.999998 |
| MAP3K3_HRD | 13 | −0.52472 | 0.43788 | 0.73569 | 0.999998 |
| AXL_DFG | 13 | −0.77849 | 0.96945 | 0.98837 | 0.999998 |
| WEE1_DFG | 11 | −0.99895 | 0.99985 | 0.99986 | 0.999998 |
| Ctrl_NONESS_PRSS54 | 18 | −0.86419 | 0.44646 | 0.80392 | 0.999998 |
| Ctrl_ESS_RPL4 | 22 | −0.49547 | 0.95916 | 0.99593 | 0.999998 |
| MAP2K1_HRD | 13 | −0.68129 | 0.38986 | 0.70108 | 0.999998 |

| id | pos. rank | pos. goodsgrna | pos. lfc |
|---|---|---|---|
| RIPK2_VAIK | 2536 | 1 | −1.0661 |
| RYK_DFG | 2477 | 2 | −1.2509 |
| MAP3K9_HRDplus5 | 2568 | 0 | −0.98684 |
| MAP2K2_VAIK | 2421 | 1 | −0.94366 |
| Ctrl_ESS_PHB2 | 1831 | 9 | −0.51128 |
| PIK3C3_HRD | 2563 | 0 | −1.1199 |
| KSR1_splice | 1615 | 8 | −1.035 |
| ADCK1_VAIK | 1329 | 2 | −1.0004 |
| ERN1_VAIK | 2566 | 0 | −0.91263 |
| PRKDC_VAIK | 2271 | 4 | −0.84195 |
| ITPKC_HRD | 2573 | 0 | −0.96916 |
| CDK16_VAIK | 2372 | 2 | −1.0981 |
| Ctrl_NONESS_BPIFB3 | 2499 | 3 | −0.61231 |
| MAP3K7_HRD | 2564 | 0 | −0.84379 |
| PRAG1_VAIK | 1890 | 2 | −0.9534 |
| CDK5_HRD | 2247 | 1 | −1.1121 |
| OBSCN_KD2_VAIK | 1914 | 1 | −0.84417 |
| SPEG_splice | 2431 | 5 | −0.61456 |
| BUB1B_HRD | 2313 | 3 | −1.0849 |
| ACVRL1_splice | 2532 | 3 | −0.80064 |
| TRIB3_splice | 1517 | 6 | −0.74447 |
| RPS6KL1_VAIK | 2174 | 4 | −0.81877 |
| Ctrl_ESS_PSMB4 | 2167 | 4 | −0.60101 |
| TESK1_VAIK | 1925 | 4 | −0.81354 |
| TRIB1_VAIK | 2515 | 1 | −0.61135 |
| Ctrl_ESS_PUF60 | 2523 | 4 | −0.77781 |
| Ctrl_NONESS_CASP2 | 2009 | 5 | 0.60753 |
| BRSK1_HRD | 656 | 4 | −0.9436 |
| DCLK1_HRDplus5 | 2514 | 2 | −0.46293 |
| TRIB3_VAIK | 2533 | 1 | −0.91471 |
| MAPK15_HRDplus5 | 1190 | 4 | −0.87391 |
| CDK9_HRD | 1765 | 2 | −0.88412 |
| AATK_HRD | 2357 | 1 | −0.60606 |
| RPS6KA5_KD2_HRD | 2405 | 2 | −1.2871 |
| EIF2AK3_VAIK | 2203 | 2 | −0.70741 |
| PRKCB_splice | 2460 | 2 | −0.79291 |
| MAP2K3_HRD | 2520 | 1 | −0.70558 |
| PRKCH_VAIK | 2171 | 2 | −0.75152 |
| MAPK14_VAIK | 1853 | 3 | −0.99297 |
| PIM3_splice | 2542 | 1 | −0.53147 |
| ADCK5_DFG | 2539 | 1 | −0.94426 |
| ALPK2_HRD | 2455 | 2 | −0.8112 |
| TAOK2_HRD | 2471 | 1 | −0.96455 |
| Ctrl_ESS_ATP6V0C | 2373 | 1 | −0.53237 |
| MAP3K3_HRD | 1784 | 1 | −0.52472 |
| AXL_DFG | 2488 | 2 | −0.77849 |
| WEE1_DFG | 2570 | 0 | −0.99895 |
| Ctrl_NONESS_PRSS54 | 1795 | 6 | −0.86419 |
| Ctrl_ESS_RPL4 | 2473 | 4 | −0.49547 |
| MAP2K1_HRD | 1694 | 1 | −0.68129 |

What is claimed is:

1. A method of modifying a nucleotide in a kinase gene on a double-stranded DNA molecule in a mammalian cell so as to modify kinase catalytic activity, the method comprising introducing to the cell a composition comprising
a) a guide RNA molecule comprising a spacer sequence portion comprising any one of SEQ ID NOs: 1-10012; and
b) a fusion protein comprising a Clustered Regularly Interspaced Short Palindromic Repeats (CRISPR) nuclease and a base editing enzyme.

2. The method of claim 1, wherein the base editing enzyme is an adenine deaminase.

3. The method of claim 1, wherein the base editing enzyme is ABE8e.

4. The method of claim 1, wherein the base editing enzyme is a cytosine deaminase, and the fusion protein preferably further comprises an inhibitor of uracil DNA glycosylase (UGI).

5. The method of claim 1, wherein the CRISPR nuclease is a nickase and effects a single-strand break in a strand of the double-stranded DNA molecule, preferably in a strand that the spacer sequence portion of the guide RNA molecule is hybridized to.

6. The method of claim 1, wherein the kinase gene is selected from the group consisting of AAK1, AATK, ABL1, ABL2, ACAD10, ACAD11, ACVR1, ACVR1B, ACVR1C, ACVR2A, ACVR2B, ACVRL1, ADCK1, ADCK2, ADCK5, AKT1, AKT2, AKT3, ALK, ALPK1, ALPK2, ALPK3, AMHR2, ANKK1, ARAF, ATM, ATR, AURKA, AURKB, AURKC, AXL, BLK, BMP2K, BMPR1A, BMPR1B, BMPR2, BMX, BRAF, BRSK1, BRSK2, BTK, BUB1, BUB1B, CASMK1, CASMK1D, CAMK1G, CAMK2A, CAMK2B, CAMK2D, CAMK2G, CAMK4, CAMKK1, CAMKK2, CAMKV, CASK, CDC42BPA, CDC42BPB, CDC42BPG, CDC7, CDK1, CDK10, CDK11A, CDK11B, CDK12, CDK13, CDK14, CDK15, CDK16, CDK17, CDK18, CDK19, CDK2, CDK20, CDK3, CDK4, CDK5, CDK6, CDK7, CDK8, CDK9, CDK1, CDKL2, CDKL3, CDKL4, CDKL5, CHEK1, CHEK2, CHKA, CHKB, CHUK, CILK1, CIT, CLK1, CLK2, CLK3, CLK4, COQ8A, COQ8B, CSF1R, CSK, CSNK1A1, CSNK1A1 L, CSNK1D, CSNK1E, CSNK1G1, CSNK1G2, CSNK1G3, CSNK2A1, CSNK2A2, CSNK2A3, DAPK1, DAPK2, DAPK3, DCLK1, DCLK2, DCLK3, DDR1, DDR2, DMPK, DSTYK, DYRK1A, DYRK1B, DYRK2, DYRK3, DYRK4, EEF2K, EGFR, EIF2AK1, EIF2AK2, EIF2AK3, EIF2AK4, EPHA1, EPHA2, EPHA3, EPHA4, EPHA5, EPHA6, EPHA7, EPHA8, EPHB1, EPHB2, EPHB3, EPHB4, ERBB2, ERBB3, ERBB4, ERN1, ERN2, ETNK1, ETNK2, FAM20A, FAM20B, FAM20C, FER, FES, FGFR1, FGFR2, FGFR3, FGFR4, FGR, FLT1, FLT3, FLT4, FN3K, FN3KRP, FRK, FYN, GAK, GRK1, GRK2, GRK3, GRK4, GRK5, GRK6, GRK7, GSK3A, GSK3B, GUCY2C, GUCY2D, GUCY2F, HASPIN, HCK, HIPK1, HIPK2, HIPK3, HIPK4, HUNK, HYKK, IGF1R, IKBKB, IKBKE, ILK, INSR, INSRR, IP6K1, IP6K2, IP6K3, IPMK, IPPK, IRAK1, IRAK2, IRAK3, IRAK4, ITK, ITPKA, ITPKB, ITPKC, JAK1, JAK2, JAK3, KALRN, KDR, KIT, KSR1, KSR2, LATS1, LATS2, LCK, LIMK1, LIMK2, LMTK2, LMTK3, LRRK1, LRRK2, LTK, LYN, MAK, MAP2K1, MAP2K2, MAP2K3, MAP2K4, MAP2K5, MAP2K6, MAP2K7, MAP3K1, MAP3K10, MAP3K11, MAP3K12, MAP3K13, MAP3K14, MAP3K15, MAP3K19, MAP3K2, MAP3K20, MAP3K21, MAP3K3, MAP3K4, MAP3K5, MAP3K6, MAP3K7, MAP3K8, MAP3K9, MAP4K1, MAP4K2, MAP4K3, MAP4K4, MAP4K5, MAPK1, MAPK10, MAPK11, MAPK12, MAPK13, MAPK14, MAPK15, MAPK3, MAPK4, MAPK6, MAPK7, MAPK8, MAPK9, MAPKAPK2, MAPKAPK3, MAPKAPK5, MARK1, MARK2, MARK3, MARK4, MAST1, MAST2, MAST3, MAST4, MASTL, MATK, MELK, MERTK, MET, MINK1, MKNK1, MKNK2, MLKL, MOK, MOS, MST1R, MTOR, MUSK, MYLK, MYLK2, MYLK3, MYLK4, MYO3A, MYO3B, NEK1, NEK10, NEK11, NEK2, NEK3, NEK4, NEK5, NEK6, NEK7, NEK8, NEK9, NIM1K, NLK, NPR1, NPR2, NRK, NTRK1, NTRK2, NTRK3, NUAK1, NUAK2, OBSCN, OXSR1, PAK1, PAK2, PAK3, PAK4, PAK5, PAK6, PASK, PBK, PDGFRA, PDGFRB, PDIK1L, PDPK1, PEAK1, PEAK3, PHKG1, PHKG2, PI4K2A, PI4K2B, PI4KA, PI4KB, PIK3C2A, PIK3C2B, PIK3C2G, PIK3C3, PIK3CA, PIK3CB, PIK3CD, PIK3CG, PIK3R4, PIKFYVE, PIM1, PIM2, PIM3, PINK1, PIP4K2A, PIP4K2B, PIP4K2C, PIP5K1A, PIP5K1B, PIP5K1C, PIP5KL1, PKDCC, PKMYT1, PKN1, PKN2, PKN3, PLK1, PLK2, PLK3, PLK4, PNCK, POMK, PRAG1, PRKAA1, PRKAA2, PRKACA, PRKACB, PRKACG, PRKCA, PRKCB, PRKCD, PRKCE, PRKCG, PRKCH, PRKCI, PRKCQ, PRKCZ, PRKD1, PRKD2, PRKD3, PRKDC, PRKG1, PRKG2, PRKX, PRPF4B, PSKH1, PSKH2, PTK2, PTK2B, PTK6, PTK7, PXK, RAF1, RET, RIOK1, RIOK2, RIOK3, RIPK1, RIPK2, RIPK3, RIPK4, RNASEL, ROCK1, ROCK2, ROR1, ROR2, ROS1, RPS6KA1, RPS6KA2, RPS6KA3, RPS6KA4, RPS6KA5, RPS6KA6, RPS6KB1, RPS6KB2, RPS6KC1, RPS6KL1, RSKR, RYK, SBK1, SBK2, SBK3, SELENOO, SGK1, SGK2, SGK3, SIK1, SIK2, SIK3, SLK, SMG1, SNRK, SPEG, SRC, SRMS, SRPK1, SRPK2, SRPK3, STK10, STK11, STK16, STK17A, STK17B, STK24, STK25, STK26, STK3, STK31, STK32A, STK32B, STK32C, STK33, STK35, STK36, STK38, STK38L, STK39, STK4, STK40, STKLD1, STYK1, SYK, TAOK1, TAOK2, TAOK3, TBCK, TBK1, TEC, TEK, TESK1, TESK2, TEX14, TGFBR1, TGFBR2, TIE1, TLK1, TLK2, TNIK, TNK1, TNK2, TNNI3K, TP53RK, TRIB1, TRIB2, TRIB3, TRIO, TRPM6, TRPM7, TSSK1B, TSSK2, TSSK3, TSSK4, TSSK6, TTBK1, TTBK2, TTK, TTN, TXK, TYK2, TYRO3, UHMK1, ULK1, ULK2, ULK3, ULK4, VRK1, VRK2, VRK3, WEE1, WEE2, WNK1, WNK2, WNK3, WNK4, YES1, and ZAP70.

7. The method of claim 1, wherein the kinase gene is selected from the group consisting of ATM, PLK4, and CDK9.

8. The method of claim 1, wherein the spacer sequence portion comprises SEQ ID NO: 6982.

9. The method of claim 1, wherein the guide RNA molecule is a single guide RNA (sgRNA) molecule.

10. A modified mammalian cell obtained by the method of claim 1.

11. A composition comprising a guide RNA molecule comprising a spacer sequence portion, wherein the spacer sequence portion comprises any one of SEQ ID NOs: 1-10012.

12. The composition of claim 11, wherein the guide RNA molecule is a crRNA molecule or a sgRNA molecule.

13. The composition of claim 11, wherein the guide RNA molecule is a crRNA molecule and the composition further comprises a tracrRNA molecule that hybridizes to a repeat sequence portion of the crRNA molecule to form a crRNA:tracrRNA complex.

14. The composition of claim 11, wherein the composition further comprises a CRISPR nuclease or a fusion protein comprising a CRISPR nuclease and a base editing enzyme.

15. The composition of claim 14, wherein the CRISPR nuclease is a nickase.

16. A plurality of modified cells, wherein the cells are modified by introducing the composition of claim 11 to the cells.

17. An isogenic cell line generated from a cell isolated from the plurality of modified cells of claim 16.

18. A method of identifying a drug target for use in a combination therapy with a first treatment, the method comprising
i) exposing the plurality of modified cells of claim 16 to the first treatment;
ii) sequencing the spacer sequence portions in the plurality of cells, preferably at a time point 0.5-14 days after the exposure to the first treatment;
iii) determining if each spacer sequence portion was enriched, unchanged, or depleted;
iv) classifying a kinase as a growth-suppressive kinase if a spacer sequence portion targeting the kinase is enriched, and classifying a kinase as an essential kinase if a spacer sequence portion targeting the kinase is depleted; and
v) identifying an essential kinase as a drug target for use in a combination therapy with the first treatment.

19. A method of identifying a drug target, the method comprising
i) sequencing the spacer sequence portions in the plurality of modified cells of claim 16, preferably at a time point 0.5-30 days after the introduction of the CRISPR guide RNA library to the plurality of cells;
ii) determining if each spacer sequence portion was enriched, unchanged, or depleted;
iii) classifying a kinase as a growth-suppressive kinase if a spacer sequence portion targeting the kinase is enriched, and classifying a kinase as an essential kinase if a spacer sequence portion targeting the kinase is depleted; and
iv) identifying an essential kinase as a drug target.

20. A method of identifying a drug target, the method comprising
i) sequencing the spacer sequence portions in the plurality of modified cells of claim 16, preferably at a time point 0.5-30 days after the introduction of the CRISPR guide RNA library to the plurality of cells;
ii) performing single-cell RNA sequencing on a cell of the plurality of modified cells, measuring gene expression levels in the cell; and
identifying a gene whose expression is altered relative to an unmodified cell as a drug target.
